SEQUENCE LISTING (58 sequences)

SEQ ID NO: 1 - protein, 8 aa, Artificial Sequence
  Synthetic peptide - insulin B16-23
YLVCGERG

SEQ ID NO: 2 - protein, 8 aa, Artificial Sequence
  Synthetic peptide - rIAg7 amino acids 73-80
AELDTACR

SEQ ID NO: 3 - protein, 15 aa, Artificial Sequence
  Synthetic peptide - insulin B9-23
SHLVEALYLVCGERG

SEQ ID NO: 4 - protein, 185 aa, Artificial Sequence
  Synthetic peptide - rIAg7 RTL
MGGDSERHFVHQFKGECYFTNGTQRIRLVTRYIYNREEYLRFDSDVGEYRAVTELGRHSAEYYNKQYLERTRAELDTACRHNYEETEVPTSLRRLGGEDDIEADHVGFYGTTVYQSPGDIGQYTHEFDGDELFYVDLDKKKTVWRLPEFGQLILFEPQGGLQNIAAEKHNLGILTKRSNFTPATN

SEQ ID NO: 5 - protein, 21 aa, Artificial Sequence
  Synthetic peptide - human MOG35-55
MEVGWYRSPFSRVVHLYRNGK

SEQ ID NO: 6 - protein, 21 aa, Artificial Sequence
  Synthetic peptide - mouse MOG35-55
MEVGWYRPPFSRVVHLYRNGK

SEQ ID NO: 7 - protein, 15 aa, Artificial Sequence
  Synthetic peptide - C18 insulin B9-23
SHLVEALYLCAGERG

SEQ ID NO: 8 - protein, 15 aa, Artificial Sequence
  Synthetic peptide - C20 insulin B9-23
SHLVEALYLVACERG

SEQ ID NO: 9 - protein, 15 aa, Artificial Sequence
  Synthetic peptide - C19A insulin B9-23
SHLVEALYLVAGERG

SEQ ID NO: 10 - protein, 11 aa, Artificial Sequence
  Synthetic peptide - rIAg7 amino acids 15-25
GECYFTNGTQR

SEQ ID NO: 11 - protein, 180 aa, Artificial Sequence
  Synthetic peptide - DR2 RTL
MGDTRPRFLWQPKRECHFFNGTERVRFLDRYFYNQEESVRFDSDVGEFRAVTELGRPDAEYWNSQKDILEQARAAVDTYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 12 - protein, 21 aa, Artificial Sequence
  Synthetic peptide - hMOG35-55 S42C
MEVGWYRCPFSRVVHLYRNGK

SEQ ID NO: 13 - protein, 21 aa, Artificial Sequence
  Synthetic peptide - hMOG35-55 P43C
MEVGWYRSCFSRVVHLYRNGK

SEQ ID NO: 14 - protein, 187 aa, Artificial Sequence
  Synthetic peptide - rIAb RTL
MGGDSERHFVYQFMGECYFTNGTQRIRYVTRYIYNREEYVRYDSDVGEHRAVTELGRPDAEYWNSQPEILERTRAELDTVCRHNYEGPETHTSLRRLGGEDDIEADHVGTYGISVYQSPGDIGQYTFEFDGDELFYVDLDKKETVWMLPEFGQLASFDPQGGLQNIAVVKHNLGVLTKRSNSTPATN

SEQ ID NO: 15 - protein, 21 aa, Artificial Sequence [sequence omitted]

SEQ ID NO: 16 - protein, 13 aa, Artificial Sequence
  Synthetic peptide - PLP139-151
HCLGKWLGHPDKF

SEQ ID NO: 17 - protein, 14 aa, Artificial Sequence
  Synthetic peptide - GAD 207-220
YEIAPVFVLLEYVT

SEQ ID NO: 18 - protein, 15 aa, Artificial Sequence
  Synthetic peptide - HEL 11-25
AMKRHGLDNYRGYSL

SEQ ID NO: 19 - protein, 178 aa, Artificial Sequence
  Synthetic polypeptide - DP2 RTL
MGDTPENYLFQGRQECYAFNGTQRFLERYIYNREEFVRFDSDVGEFRAVTELGRPDEEYWNSQKDILEEERAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 20 - protein, 183 aa, Artificial Sequence [sequence omitted]

SEQ ID NO: 21 - protein, 25 aa, Artificial Sequence
  Synthetic peptide - MOG 1-25
GQFRVIGPRHPIRALVGDEVELPCR

SEQ ID NO: 22 - protein, 23 aa, Artificial Sequence
  Synthetic peptide - MOG94-116
GGFTCFFRDHSYQEEAAMELKVE

SEQ ID NO: 23 - protein, 16 aa, Artificial Sequence
  Synthetic peptide - MOG 145-160
VFLCLQYRLRGKLRAE

SEQ ID NO: 24 - protein, 15 aa, Artificial Sequence
  Synthetic peptide - MOG 194-208
LVALIICYNWLHRRL

SEQ ID NO: 25 - protein, 21 aa, Artificial Sequence
  Synthetic peptide - MBP 10-30
RHGSKYLATASTMDHARHGFL

SEQ ID NO: 26 - protein, 11 aa, Artificial Sequence
  Synthetic peptide - MBP 35-45
DTGILDSIGRF

SEQ ID NO: 27 - protein, 14 aa, Artificial Sequence
  Synthetic peptide - MBP 77-91
SHGRTQDENPVVHF

SEQ ID NO: 28 - protein, 15 aa, Artificial Sequence
  Synthetic peptide - MBP 85-99
ENPVVHFFKNIVTPR

SEQ ID NO: 29 - protein, 18 aa, Artificial Sequence
  Synthetic peptide - MBP 95-112
IVTPRTPPPSQGKGRGLS

SEQ ID NO: 30 - protein, 20 aa, Artificial Sequence
  Synthetic peptide - MBP 145-164
VDAQGTLSKIFKLGGRDSRS

SEQ ID NO: 31 - protein, 22 aa, Artificial Sequence
  Synthetic peptide - PLP 95-116
GAVRQIFGDYKTTICGKGLSAT

SEQ ID NO: 32 - protein, 14 aa, Artificial Sequence
  Synthetic peptide - type II collagen 261-274
AGFKGEQGPKGEPG

SEQ ID NO: 33 - protein, 15 aa, Artificial Sequence
  Synthetic peptide - type II collagen 259-273
GIAGFKGEQGPKGEP

SEQ ID NO: 34 - protein, 14 aa, Artificial Sequence
  Synthetic peptide - type II collagen 257-270
EPGIAGFKGEQGPK

SEQ ID NO: 35 - protein, 14 aa, Artificial Sequence
  Synthetic peptide - type II collagen 257-270 modified
APGIAGFKAEQAAK

SEQ ID NO: 36 - protein, 15 aa, Artificial Sequence
  Synthetic peptide - IRBP 1177-1191
ADGSSWEGVGVVPDV

SEQ ID NO: 37 - protein, 20 aa, Artificial Sequence
  Synthetic peptide - arrestin 291-310
NRERRGIALDGKIKHEDTNL

SEQ ID NO: 38 - protein, 32 aa, Artificial Sequence
  Synthetic peptide - phosducin 65-96
KERMSRKMSIQEYELIHQDKEDEGCLRKYRRQ

SEQ ID NO: 39 - protein, 5 aa, Artificial Sequence
  Synthetic peptide - recoverin 48-52
QFQSI

SEQ ID NO: 40 - protein, 7 aa, Artificial Sequence
  Synthetic peptide - recoverin 64-70
KAYAQHV

SEQ ID NO: 41 - protein, 20 aa, Artificial Sequence
  Synthetic peptide - recoverin 62-81
PKAYAQHVFRSFDANSDGTL

SEQ ID NO: 42 - protein, 14 aa, Artificial Sequence
  Synthetic peptide - recoverin 149-162
EKRAEKIWASFGKK

SEQ ID NO: 43 - protein, 20 aa, Artificial Sequence
  Synthetic peptide - fibrinogen alpha 40-59
VERHQSACKDSDWPFCSDED

SEQ ID NO: 44 - protein, 20 aa, Artificial Sequence
  Synthetic peptide - fibrinogen alpha - 616-625
THSTKRGHAKSRPVRGIHTS

SEQ ID NO: 45 - protein, 13 aa, Artificial Sequence
  Synthetic peptide - fibrinogen alpha 79-91
QDFTNRINKLKNS

SEQ ID NO: 46 - protein, 20 aa, Artificial Sequence
  Synthetic peptide - fibrinogen alpha 121-140
NNRDNTYNRVSEDLRSRIEV

SEQ ID NO: 47 - protein, 21 aa, Artificial Sequence
  Synthetic peptide - vimentin 59-79
GVYATRSSAVRLRSSVPGVRL

SEQ ID NO: 48 - protein, 19 aa, Artificial Sequence
  Synthetic peptide - vimentin 26-44
SSRSYVTTSTRTYSLGSAL

SEQ ID NO: 49 - protein, 20 aa, Artificial Sequence
  Synthetic peptide - vimentin 256-275
IDVDVSKPDLTAALRDVRQQ

SEQ ID NO: 50 - protein, 19 aa, Artificial Sequence
  Synthetic peptide - vimentin 415-433
LPNFSSLNLRETNLDSLPL

SEQ ID NO: 51 - protein, 17 aa, Artificial Sequence
  Synthetic peptide - alpha enolase 5-21
KIHAREIFDSRGNPTVE

SEQ ID NO: 52 - protein, 13 aa, Artificial Sequence
  Synthetic peptide - human cartilage glycoprotein 39 259-271
PTFGRSFTLASSE

SEQ ID NO: 53 - protein, 11 aa, Artificial Sequence
  Synthetic peptide - alpha2 gliadin 61-71
FPQPELPYPQP

SEQ ID NO: 54 - protein, 20 aa, Artificial Sequence
  Synthetic peptide - alpha2 gliadin 58-77
LQPFPQPQLPYPQPQLPYPQ

SEQ ID NO: 55 - protein, 180 aa, Artificial Sequence
  Synthetic polypeptide - DR3 RTL
MGDTRPRFLEYSTSECHFFNGTERVRYLDRYFHNQEENVRFDSDVGEFRAVTELGRPDAEYWNSQKDLLEQKRGRVDNYCRHNYGVVESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 56 - protein, 180 aa, Artificial Sequence
  Synthetic polypeptide - DR4 RTL
MGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 57 - protein, 185 aa, Artificial Sequence
  Synthetic polypeptide - rIAs RTL
MGGDSERHFVFQFKGECYFTNGTQRIRSVDRYIYNREEYLRFDSDVGEYRAVTELGRPDAEYYNKQYLEQTRAELDTVCRHNYEGVETHTSLRRLGGQDDIEADHVGVYGTTVYQSPGDIGQYTHEFDGDEWFYVDLDKKETIWMLPEFGQLTSFDPQGGLQNIATGKYTLGILTKRSNSTPATN

SEQ ID NO: 58 - protein, 185 aa, Artificial Sequence
  Synthetic polypeptide - rat RT1.B RTL
MGRDSPRDFVYRFKGLCYYTNGTQRIRDVIRYIYNQEEYLRYDSDVGEYRALTELGRPSAEYFNKQYLEQTRAELDTVCRHNYEGSEVRTSLRRLGGQDDIEADHVAAYGINMYQYYESRGQFTHEFDGDEEFYVDLDKKETIWRIPEFGQLTSFDPQGGLQNIAIIKHNLEILMKRSNSTQAVN

(19) United States
(12) Patent Application Publication
(10) Pub. No.: US 2013/0171179 A1
(43) Pub. Date: Jul. 4, 2013

(54) RECOMBINANT T-CELL RECEPTOR LIGANDS WITH COVALENTLY BOUND PEPTIDES

(71) Applicant: Gregory G. Burrows, Portland, OR (US)

(72) Inventor: Gregory G. Burrows, Portland, OR (US)

(73) Assignee: Oregon Health & Science University, Portland, OR (US)

(21) Appl. No.: 13/820,393

(22) PCT Filed: Sep. 2, 2011
(86) PCT No.: PCT/US11/50419

§ 371 Date: Mar. 1, 2013

Related U.S. Application Data
(60) Provisional application No. 61/380,191, filed on Sep. 3, 2010.

(51) Int. Cl. C07K 14/74; C07K 1/107; A61K 47/48

(52) U.S. Cl. CPC C07K 14/70539; A61K 47/48276; C07K 1/1075
USPC 424/185.1; 530/408; 424/194.1; 252/182.3

(57) ABSTRACT

Disclosed herein are stable complexes including an MHC class I or MHC class II recombinant T cell receptor ligand RTL polypeptide covalently linked to an antigenic determinant by a disulfide bond. Also disclosed are methods of making such compositions and methods of use, for example to treat or inhibit a disorder, for example, an autoimmune disorder.

### CROSS REFERENCE TO RELATED APPLICATION

This claims the benefit of U.S. Provisional Application No. 61/380,191, filed Sep. 3, 2010, which is incorporated herein by reference in its entirety.

### ACKNOWLEDGMENT OF GOVERNMENT SUPPORT

This invention was made with government support under grant numbers AI43960 and DK068881 awarded by the National Institutes of Health. The government has certain rights in the invention.

### FIELD

This disclosure relates to compositions including major histocompatibility complex (MHC) polypeptides covalently linked to peptide antigens and methods utilizing these compositions, for example to modulate an immune response.

### BACKGROUND

The initiation of an immune response against a specific antigen in mammals is brought about by the presentation of that antigen to T-cells. An antigen is presented to T-cells in the context of a major histocompatibility (MHC) complex. MHC complexes are located on the surface of antigen presenting cells (APCs); the three-dimensional structure of MHCs includes a groove or cleft into which the presented antigen fits. When an appropriate receptor on a T-cell interacts with the MHC/antigen complex on an APC in the presence of necessary co-stimulatory signals, the T-cell is stimulated, triggering various aspects of the well-characterized cascade of immune system activation events, including induction of cytotoxic T-cell function, induction of B-cell activity, and stimulation of cytokine production.

There are two basic classes of MHC molecules in mammals, MHC class I and MHC class II. Both classes are large protein complexes formed by association of two separate proteins. Each class includes transmembrane domains that anchor the complex into the cell membrane. MHC class I molecules are formed from two non-covalently associated proteins, the α chain and β2-microglobulin. The α chain comprises three distinct domains, α1, α2, and α3. The three-dimensional structure of the α1 and α2 domains forms the groove into which antigens fit for presentation to T-cells. The α3 domain is an Ig-fold like domain that contains a transmembrane sequence that anchors the α chain into the cell membrane of the APC. MHC class I complexes, when associated with antigen (and in the presence of appropriate co-stimulatory signals) stimulate CD8 cytotoxic T-cells, which function to kill any cell which they specifically recognize.

The two proteins which associate non-covalently to form MHC class II molecules are termed the α and β chains. The α chain comprises α1 and α2 domains, and the β chain comprises β1 and β2 domains. The cleft into which the antigen fits is formed by the interaction of the α1 and β1 domains. The α2 and β2 domains are transmembrane Ig-fold like domains that anchor the α and β chains into the cell membrane of the APC. MHC class II complexes, when associated with antigen (and in the presence of appropriate co-stimulatory signals) stimulate CD4 T-cells. CD4 T-cells serve a myriad of purposes within the immune system, including initiating an inflammatory response, regulating other immune cells, providing help to B cells for antibody synthesis, modulating the immune response so the appropriate immune response to a given pathogen is achieved, secreting cytokines, and/or expressing membrane bound factors, among others.

The role that MHC complexes play in the immune system has led to the development of methods by which these complexes are used to modulate the immune response. For example, activated T-cells that recognize “self” antigenic peptides (autoantigens) in the context of MHC are known to play a key role in autoimmune diseases such as rheumatoid arthritis and multiple sclerosis. Because isolated MHC class II molecules (loaded with the appropriate antigen) can substitute for APCs carrying the MHC class II complex and can bind to antigen-specific T-cells, many have proposed that isolated MHC/antigen complexes may be used to treat autoimmune disorders. (See U.S. Pat. Nos. 5,194,425 and 5,284,935).

In another context, it has been shown that the interaction of isolated MHC II/antigen complexes with T-cells, in the absence of co-stimulatory factors, induces a state of nonresponsiveness known as anergy. (Quill et al, J. Immunol., 138:3704-3712, 1987). Following this observation, Sharma et al. (U.S. Pat. Nos. 5,468,481 and 5,130,297) and Clarke et al. (U.S. Pat. No. 5,260,422) have suggested that such isolated MHC II/antigen complexes may be administered therapeutically to anergize T-cell lines that specifically respond to particular autoantigenic peptides.

### SUMMARY

Although the concept of using isolated MHC/antigen complexes in therapeutic and diagnostic applications holds great promise, a major drawback to the various methods reported to date is that the complexes are large and consequently difficult to produce and to work with. It is shown herein that recombinant MHC polypeptides (such as recombinant two domain MHC class I or MHC class II polypeptides) can be covalently linked to a peptide antigen to produce a stable complex. Disclosed herein are stable complexes including an MHC class I or MHC class II recombinant T cell receptor ligand (RTL) polypeptide covalently linked to an antigenic determinant by a disulfide bond. Also disclosed are methods of making such compositions and methods of use, for example to treat or inhibit an autoimmune disease.

In some embodiments, the disclosed compositions include a recombinant MHC polypeptide including covalently linked first and second domains wherein the first domain is a mammalian MHC class II β1 domain and the second domain is a mammalian MHC class II α1 domain, wherein the amino terminus of the α1 domain is covalently linked to the carboxy terminus of the β1 domain and wherein the MHC class II polypeptide does not include an α2 or a β2 domain, and an antigenic determinant (such as a peptide antigen) covalently linked to the recombinant MHC polypeptide by a disulfide bond. In other embodiments, the disclosed compositions include a recombinant MHC polypeptide including covalently linked first and second domains wherein the first domain is a mammalian MHC class I α1 domain and the second domain is a mammalian MHC class I α2 domain, wherein the amino terminus of the α2 domain is covalently linked to the carboxy terminus of the α1 domain and wherein the MHC class I polypeptide does not include an α3 domain, and an antigenic determinant (such as a peptide antigen) covalently linked to the recombinant MHC polypeptide by a disulfide bond. In some examples, the recombinant MHC polypeptide has reduced potential for aggregation in solution, for example, a recombinant MHC polypeptide including substitution of one or more hydrophobic amino acids in a β-sheet platform of the MHC polypeptide with a polar or charged amino acid.

In some examples, the disulfide linkage is formed utilizing a naturally occurring cysteine residue in the MHC polypeptide (such as a cysteine residue in the MHC class II β1 domain or a cysteine residue in an MHC class I α domain). In other examples, the disulfide linkage is formed utilizing a non-naturally occurring cysteine residue in the MHC polypeptide, such as a cysteine residue introduced in the MHC polypeptide by mutagenesis. In further examples, the disulfide linkage is formed utilizing a naturally occurring cysteine residue in the antigenic determinant. In still further examples, the disulfide linkage is formed utilizing a non-naturally occurring cysteine residue in the peptide antigen, such as a cysteine residue introduced in the antigenic determinant by mutagenesis.

Also disclosed herein are methods of making the disclosed compositions and kits for producing the disclosed compositions. In some examples, the methods include use of a buffer or solution including one or more components for facilitating (for example providing conditions sufficient for) formation of a disulfide bond between the recombinant MHC polypeptide and the antigenic determinant.

Methods of using the disclosed compositions are also provided herein. In some embodiments, the methods include treating or inhibiting an autoimmune disease in a subject including administering an effective amount of a composition including a recombinant MHC polypeptide disclosed herein covalently linked to an antigenic determinant by a disulfide bond. In particular examples, the autoimmune disease includes, but is not limited to multiple sclerosis, type I diabetes, rheumatoid arthritis, celiac disease, or psoriasis.

The foregoing and other features of the disclosure will become more apparent from the following detailed description, which proceeds with reference to the accompanying figures.

### BRIEF DESCRIPTION OF THE DRAWINGS

FIG. 1A is a digital image of a Coomassie blue stained 10-20% SDS-PAGE showing “empty” rIAg7 (−) and rIAg7 bearing disulfide captured insulin B:9-23 peptide (WT). rIAg7/peptide migrates as higher molecular weight species (29, 31 and 33 kD). Samples were loaded and treatment conditions were as indicated (Red, reducing; NR, non-reducing).

FIG. 1B is a bar graph showing quantitation of the bands shown in lanes 7 and 8 of FIG. 1A.

FIG. 2 is a digital image showing a comparison of capture by rIAg7 of FITC-labeled insulin B:9-23 peptide and variants, as indicated. Wild-type insulin B:9-23 peptide (C19) was the most efficiently captured by rIAg7. Insulin B:9-23 variants with the cysteine moved toward the amino-terminal end (to position 18; C18) or toward the carboxyl-terminal end (to position 20; C20) were captured, but with much less efficiency, even after 50 hours incubation, as shown. Peptide sequences are shown below the digital image.

FIG. 3 is a pair of digital images showing time course of peptide capture by rIAg7. Insulin B:16-23 peptide (FITC-YLVCGERG; SEQ ID NO: 1) and rIA7 were mixed (10:1, peptide:rIAg7) in 100 mM NaPO₄, pH 6.5, 150 mM NaCl, 0.05% SDS and 0.01% NaN₃ for the indicated times.

FIG. 4 is a graph showing densitometry results for the 29 kD and 31 kD bands of the time course shown in FIG. 3. Coomassie stained bands were quantified to determine an initial rate of capture.

FIG. 5 is a pair of mass spectrometry plots of whole mass measurements of rIAg7 showing presence of an internal disulfide bond. Samples of rIAg7 were alkylated or reduced and alkylated. Alkylated rIAg7 (left) showed a primary peak at 21,416.7 Da, corresponding very closely to the expected mass of 21,420.6 for rIAg7 missing the amino terminal methionine. Reduced and alkylated rIAg7 (right) showed a primary peak at 21,530.3, corresponding very closely to the expected mass for rIAg7 missing the amino terminal methionine plus two additional alkyl groups. A secondary peak at 21,665.5 corresponds very closely to the expected mass of 21,665.8 for rIAg7 with its amino terminal methionine intact plus two alkyl groups.

FIG. 6 is a digital image of SDS-PAGE of purified rIAg7 and rIAg7 mixed with insulin B:9-23 peptide (left). Molecular weight standards are shown. The bands corresponding to rIAg7 and two higher molecular weight bands at 29 and 31 kD (middle) were cut out of the gel, digested with trypsin and analyzed by mass spectrometry. The rIAg7 band contained a disulfide-linked peptide containing the intact C17-C79 disulfide bond (right, bottom). Both the 29 kD and the 31 kD bands contained disulfide-linked peptides containing the insulin B:9-23 peptide cross-linked to the rIAg7 C79-containing peptide AELDTACR (SEQ ID NO: 2) (right, top).

FIG. 7 is a digital image of SDS-PAGE of purified recombinant human DR2 (−) loaded with MOG35-55 (WT), MOG35-55 S42C variant, or MOG35-55 P43C variant (left). Samples were loaded and treatment conditions were as indicated (Red, reducing; NR, non-reducing). Densitometry data of lanes 5, 6, 7, and 8 (DR2, 29 kD, 31 kD, and 33 kD bands, left to right) are shown at the right.

FIG. 8 is a digital image of SDS-PAGE of recombinant murine I-A^{b} loaded with mouse MOG35-55 S45C variant. Samples were loaded and treatment conditions were as indicated (Red, reducing; NR, non-reducing).

FIG. 9 is a model of insulin B:9-23 bound to IAg7 in unconventional binding register. This binding register supports the efficient redox capture of insulin B:9-23 when Cys 19 occupies the P4 pocket, placing it at an appropriate distance from the C17-C79 intra-chain disulfide bond.

FIG. 10 is a plot showing survival of non-obese diabetic (NOD) mice treated with rIAg7 loaded with disulfide captured insulin B:9-23 (rIAg7-insulin), empty rIAg7 (RTL450), or vehicle (Tris-buffer).

FIG. 11A is a graph showing EAE score over time post-treatment in mice treated with vehicle, RTL551, or RTL550 loaded with disulfide captured MOG35-55 (RTL550-Cys-MOG).

FIG. 11B is a bar graph showing cumulative disease index in the mice treated as shown in FIG. 11A.

FIG. 12A is a graph showing EAE score over time post-treatment in gamma interferon-inducible lysosomal thiol reductase (GILT) knockout mice treated with vehicle (untreated) or RTL550 with disulfide captured MOG35-55 (RTL550-Cys-MOG).

FIG. 12B is a bar graph showing cumulative disease index in the mice treated as shown in FIG. 112.

FIG. 13A-C is a series of diagrams showing the predicted structure of MHC class II polypeptides. FIG. 13A is a model of an HLA-DR2 polypeptide on the surface of an antigen presenting cell (APC). FIG. 13B is a model of an exemplary MHC class II β1α1 molecule. FIG. 13C is a model of an exemplary β-sheet platform from a HLA-DR2 β1α1 molecule showing the hydrophobic residues.

FIG. 14 is an alignment of amino acid sequences of exemplary human, mouse, and rat MHC class II β1α1 polypeptides. * indicates gaps introduced for optimal sequence alignment. Arrow indicates the β1/α1 junction. Cysteine residues are shaded. Italics indicate non-native linker residues between β1 and α1 domains. Underlined residues are residues in RTL302 (DR2) that are substituted with serine or aspartate in modified RTLs with reduced aggregation in solution. Sequence identifiers are as follows: DR2 (SEQ ID NO: 11), DR3 (SEQ ID NO: 55), DR4 (SEQ ID NO: 56), DP2 (SEQ ID NO: 19), DQ2 (SEQ ID NO: 20), IAs (SEQ ID NO: 57), IAg7 (SEQ ID NO: 4), IAb (SEQ ID NO: 14), and RT1.B (SEQ ID NO: 58).

### SEQUENCE LISTING

The disclosed nucleic acid and amino acid sequences referenced herein are shown using standard letter abbreviations for nucleotide bases and amino acids, as defined in 37 C.F.R. 1.822. In at least some cases, only one strand of each nucleic acid sequence is shown, but the complementary strand is understood as included by any reference to the displayed strand.

The Sequence Listing is submitted as an ASCII text file in the form of the file named Sequence_Listing.txt, which was created on Sep. 2, 2011, and is 28,991 bytes, which is incorporated by reference herein.

SEQ ID NO: 1 is the amino acid sequence of an insulin B:16-23 peptide.

SEQ ID NO: 2 is the amino acid sequence of rIAg7 RTL amino acids 73-80.

SEQ ID NO: 3 is the amino acid sequence of insulin B:9-23 peptide.

SEQ ID NO: 4 is the amino acid sequence of rIAg7 RTL.

SEQ ID NO: 5 is the amino acid sequence of human myelin oligodendrocyte glycoprotein (MOG) 35-55 peptide.

SEQ ID NO: 6 is the amino acid sequence of mouse MOG35-55 peptide.

SEQ ID NO: 7 is the amino acid sequence of insulin B:9-23 C18 variant.

SEQ ID NO: 8 is the amino acid sequence of insulin B:9-23 C20 variant.

SEQ ID NO: 9 is the amino acid sequence of insulin B:9-23 C19A variant.

SEQ ID NO: 10 is the amino acid sequence of rIAg7 RTL amino acids 15-25.

SEQ ID NO: 11 is the amino acid sequence of an exemplary DR2 RTL.

SEQ ID NO: 12 is the amino acid sequence of hMOG35-55 S42C variant.

SEQ ID NO: 13 is the amino acid sequence of hMOG35-55 P43C variant.

SEQ ID NO: 14 is the amino acid sequence of rI-A^{b} RTL.

SEQ ID NO: 15 is the amino acid sequence of mMOG35-55 S45C variant.

SEQ ID NO: 16 is the amino acid sequence of proteolipid protein (PLP) 139-151 peptide.

SEQ ID NO: 17 is the amino acid sequence of glutamic acid decarboxylase (GAD) 207-220 peptide.

SEQ ID NO: 18 is the amino acid sequence of hen egg lysozyme (HEL) 11-25 peptide.

SEQ ID NO: 19 is the amino acid sequence of an exemplary DP2 RTL.

SEQ ID NO: 20 is the amino acid sequence of an exemplary DQ2 RTL.

SEQ ID NOs: 21-24 are amino acid sequences of exemplary MOG peptides.

SEQ ID NOs: 25-30 are amino acid sequences of exemplary myelin basic protein (MBP) peptides.

SEQ ID NO: 31 is the amino acid sequence of an exemplary PLP peptide.

SEQ ID NOs: 32-35 are amino acid sequences of exemplary collagen type II peptides.

SEQ ID NO: 36 is the amino acid sequence of interphotoreceptor retinoid binding protein (IRBP) 1177-1191 peptide.

SEQ ID NO: 37 is the amino acid sequence of arrestin 291-310 peptide.

SEQ ID NO: 38 is the amino acid sequence of phosducin 65-96 peptide.

SEQ ID NOs: 39-42 are amino acid sequences of exemplary recoverin peptides.

SEQ ID NOs: 43-46 are amino acid sequences of exemplary fibrinogen-α peptides.

SEQ ID NOs: 47-50 are amino acid sequences of exemplary vimentin peptides.

SEQ ID NO: 51 is the amino acid sequence of α-enolase 5-21 peptide.

SEQ ID NO: 52 is the amino acid sequence of human cartilage glycoprotein 39 259-271 peptide.

SEQ ID NOs: 53 and 54 are the amino acid sequences of exemplary α2-gliadin peptides.

SEQ ID NO: 55 is the amino acid sequence of an exemplary DR3 RTL.

SEQ ID NO: 56 is the amino acid sequence of an exemplary DR4 RTL.

SEQ ID NO: 57 is the amino acid sequence of an exemplary IAs RTL.

SEQ ID NO: 58 is the amino acid sequence of an exemplary rat RT1.B RTL.

### DETAILED DESCRIPTION

Although the concept of using isolated MHC/antigen complexes in therapeutic and diagnostic applications holds great promise, a major drawback to the various methods reported to date is that the complexes are large and consequently difficult to produce and to work with. A major breakthrough in this regard was the development of recombinant T-cell ligands, or RTLs. RTLs comprise a soluble single chain polypeptide homologous to the peptide binding domain of a class I or class II MHC molecule. A peptide may be loaded into the antigen binding cleft and the RTL-peptide complex used in any of a number of methods of modulating an immune response. A drawback of RTLs is that the peptide is bound into the antigenic cleft merely by non-covalent binding interactions and therefore the complex is relatively unstable. RTLs have also been constructed with the antigenic determinant included as a genetically encoded amino terminal extension of the recombinant MHC polypeptide. These complexes are stable, however, they must be individually designed and are time-consuming to produce.

Disclosed herein are compositions in which the antigenic determinant is covalently linked to the RTL polypeptide (for example a β1α1 MHC class II RTL polypeptide or an α1α2 MHC class I RTL polypeptide) by a disulfide bond. In the case of a β1α1 polypeptide, the disulfide bond between the RTL polypeptide and the antigenic determinant disrupts an internal disulfide bond in the β1 subunit. Surprisingly, the RTL maintains its structure and function even when this internal disulfide bond is disrupted. Furthermore, the disulfide bond provides a stable linkage between the antigenic determinant and the MHC polypeptide. This stable interaction is particularly important is pharmaceutical compositions intended for administration to a subject, both in terms of maintaining potency and efficacy, as well as satisfying regulatory criteria for such compositions. Furthermore the disclosed compositions can be quickly and conveniently produced by simply loading an RTL with a selected antigenic determinant, facilitating production and testing of such compositions. These compositions also show increased efficacy for treating or inhibiting a disease or disorder in a subject, such as an autoimmune disorder.

Some peptide antigens contain post-translational modifications (such as glycosylation or citrullination). In human rheumatoid arthritis a key target of the aberrant immune response are proteins that have undergone citrullination. Similarly, citrullination of MBP has been suggested to play an important role in the pathology of multiple sclerosis, with six sites on human MBP citrullinated in pathological settings. These modified peptide antigens cannot be genetically encoded at the amino-terminal of previously described RTLs (e.g., U.S. Pat. No. 6,270,772; U.S. Pat. Publ. No. 2005/0142142) and the chemistry described here provides a practical solution to this problem.

An additional advantage of a disulfide linkage between the antigenic determinant and MHC polypeptide is that this linkage is maintained following internalization until the complex reaches the deep endosome, where the antigenic determinant is cleaved by gamma interferon-inducible lysosomal thiol reductase (GILT). The antigenic determinant is then available to be reloaded onto full-length (native) MHC class II molecules for presentation at the cell surface, and may produce a tolerogenic response.

### I. Abbreviations and Terms

APC antigen presenting cell

EAE experimental autoimmune encephalomyelitis

FACS fluorescence activated cell sorting

GAD glutamic acid decarboxylase

GILT gamma interferon-inducible lysosomal thiol reductase

HEL hen egg lysozyme

HLA human leukocyte antigen

IAA iodoacetamide

IRBP interphotoreceptor retinoid binding protein

MBP myelin basic protein

MHC major histocompatibility complex

MOG myelin oligodendrocyte glycoprotein

NOD non-obese diabetic mouse

PLP proteolipid protein

RTL recombinant T cell receptor ligand

SDS sodium dodecyl sulfate

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments, the following explanations of certain terms are provided:

β1α1 Polypeptide: A recombinant polypeptide comprising the α1 and β1 domains of a MHC class II molecule in covalent linkage. To ensure appropriate conformation, the orientation of such a polypeptide is such that the carboxyl terminus of the β1 domain is covalently linked to the amino terminus of the α1 domain. In one non-limiting embodiment, the polypeptide is a human β1α1 polypeptide, and includes the α1 and β1 domains for a human MHC class II molecule. One specific, non-limiting example of a human β1α1 polypeptide is a molecule wherein the carboxyl terminus of the β1 domain is covalently linked to the amino terminus of the α1 domain of an HLA-DR molecule. Additional specific non-limiting examples of a human β1α1 polypeptide are a molecule wherein the carboxyl terminus of the β1 domain is covalently linked to the amino terminus of the α1 domain of an HLA-DR(either A or B), an HLA-DP(A and B), or an HLA-DQ(A and B) molecule. In one embodiment, the NaI polypeptide does not include a β2 domain. In another embodiment, the NaI polypeptide does not include an α2 domain. In yet another embodiment, the NaI polypeptide does not include either an α2 or a β2 domain. Exemplary β1α1 polypeptides are described in U.S. Pat. No. 6,270,772; U.S. Pat. Publ. No. 2005/0142142 and are provided herein (e.g., SEQ ID NOs: 4, 11, 14, 19, 20, and 55-58).

β1α1 Gene: A recombinant nucleic acid molecule including a nucleic acid sequence encoding β3α1 polypeptide. In some embodiments a β1α1 gene includes a promoter region operably linked to a nucleic acid encoding β1α1 polypeptide. In one embodiment the encoded β1α1 polypeptide is a human β1α1 polypeptide.

α1α2 Polypeptide: A polypeptide comprising the α1 and α2 domains of an MHC class I molecule in covalent linkage. The orientation of such a polypeptide is such that the carboxyl terminus of the α1 domain is covalently linked to the amino terminus of the α2 domain. An α1α2 polypeptide comprises less than the whole class I α chain, and usually omits most or all of the α3 domain of the α chain. Specific non-limiting examples of an α1α2 polypeptide are polypeptides wherein the carboxyl terminus of the α domain is covalently linked to the amino terminus of the α2 domain of an HLA-A, -B or -C molecule. In one embodiment, the α3 domain is omitted from an α1α2 polypeptide, thus the α1α2 polypeptide does not include an α3 domain.

α1α2 gene: A recombinant nucleic acid molecule including a nucleic acid sequence encoding an α1α2 polypeptide. In some embodiments an α1α2 gene includes a promoter region operably linked to a nucleic acid encoding an α1α2 polypeptide. In one embodiment the encoded α1α2 polypeptide is a human α1α2 polypeptide.

Antigen: A compound, composition, or substance that can stimulate the production of antibodies or a T-cell response in an animal, including compositions that are injected or absorbed into an animal. An antigen reacts with the products of specific humoral or cellular immunity, including those induced by heterologous immunogens. The term “antigen” includes all related antigenic epitopes and antigenic determinants, such as an antigenic peptide that is presented in the context of a recombinant MHC molecule disclosed herein.

Autoimmune disorder: A disorder in which the immune system produces an immune response (e.g., a B cell or a T cell response) against an endogenous antigen, with consequent injury to tissues. Exemplary autoimmune disorders include, but are not limited to multiple sclerosis, type I diabetes, rheumatoid arthritis, celiac disease, psoriasis, systemic lupus erythematosus, pernicious anemia, myasthenia gravis, and Addision's disease.

Conservative substitution or variant: A substitution of an amino acid residue for another amino acid residue having similar biochemical properties. A peptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples:

| Original Amino Acid | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Domain: A domain of a polypeptide or protein is a discrete part of an amino acid sequence that can be equated with a particular function. For example, the α and β polypeptides that constitute an MHC class II molecule are each recognized as having two domains, α1, α2 and β1, β2, respectively. Similarly, the α chain of MHC class I molecules is recognized as having three domains, α1, α2, and α3. The various domains in each of these molecules are typically joined by linking amino acid sequences. In one embodiment, when selecting the sequence of a particular domain for inclusion in a recombinant molecule, the entire domain is included; to ensure that this is done, the domain sequence may be extended to include part of the linker, or even part of the adjacent domain.

The precise number of amino acids in the various MHC molecule domains varies depending on the species of mammal, as well as between classes of genes within a species. Rather than a precise structural definition based on the number of amino acids, it is the maintenance of domain function that is important when selecting the amino acid sequence of a particular domain. Moreover, one of skill in the art will appreciate that domain function may also be maintained if somewhat less than the entire amino acid sequence of the selected domain is utilized. For example, a number of amino acids at either the amino or carboxyl termini of the α1 domain may be omitted without affecting domain function. Typically however, the number of amino acids omitted from either terminus of the domain sequence will be no greater than 10, and more typically no greater than 5. The functional activity of a particular selected domain may be assessed in the context of the two-domain MHC polypeptides provided by this disclosure (e.g., recombinant class II β1α1 or class I α1α2 polypeptides) using an antigen-specific T-cell proliferation assay. For example, to test a particular β1 domain, it will be linked to a functional α1 domain so as to produce a β1α1 molecule and then tested in the T cell proliferation assay. A biologically active β1α1 or α1α2 polypeptide will inhibit antigen-specific T cell proliferation by at least about 50%, thus indicating that the component domains are functional. Typically, such polypeptides will inhibit T-cell proliferation in this assay system by at least 75% and sometimes by greater than about 90%.

Effective amount: An amount of a composition or pharmaceutical preparation that alone, or together with a pharmaceutically acceptable carrier or one or more additional agents, induces the desired response. Effective amounts of an agent can be determined in many different ways, such as an improvement of physiological condition of a subject, relieving symptoms caused by a disease, or inhibiting development of a disease or condition. Effective amounts also can be determined through various in vitro, in vivo, or in situ assays.

Epitope: An antigenic determinant. These are particular chemical groups or peptide sequences on a molecule that are antigenic, e.g., that elicit a specific immune response. An antibody binds a particular antigenic epitope. In the case of T cells, a T cell epitope is a particular antigenic peptide presented in the context of an MHC molecule that is recognized by the T cell receptor. A T cell epitope that produces a particularly robust T cell response may be designated a dominant T cell epitope.

Equivalent: Polypeptides (such as an RTL or antigenic determinant) with one or more sequence alterations that yield the same or similar outcome in a given situation (such as an experimental or therapeutic setting) are considered equivalent (or functionally equivalent) polypeptides. Such sequence alterations can include, but are not limited to, conservative substitutions, deletions, mutations, frame shifts, and insertions.

Immune response: A response of the immune system to an immunogenic stimulus, such as an antigenic challenge. In one embodiment, the response is specific for a particular antigen (an “antigen-specific response”). Depending on the type of antigenic challenge (e.g., pathogen, allergen, or toxin), different types of immune response may occur. In some examples, an immune response includes Th1 responses, Th2 responses, Th3 response, Th17 responses, suppressor T cell responses, delayed type hypersensitivity responses, immediate type hypersensitivity responses, inflammatory responses, cell-mediated immune responses, specific immune responses, non-specific immune responses, innate immune responses, responses that involve one or more components of the complement system, or any other immune response.

Inhibiting or treating a disease: “Inhibiting” a disease refers to inhibiting the full development of a disease, for example in a person who is known to have a disease such as an autoimmune disorder or has a predisposition to a disease such as an autoimmune disorder. Inhibition of a disease can span the spectrum from partial inhibition to substantially complete inhibition (prevention) of the disease. In some examples, the term “inhibiting” refers to reducing or delaying the onset or progression of a disease. A subject to be administered an effective amount of the pharmaceutical compound to inhibit or treat the disease or disorder can be identified by standard diagnosing techniques for such a disorder, for example, basis of symptoms, medical history, family history, or risk factors to develop the disease or disorder. “Treatment” refers to a therapeutic intervention that ameliorates a sign or symptom of a disease or pathological condition after it has begun to develop.

Isolated: An “isolated” nucleic acid has been substantially separated or purified away from other nucleic acids (e.g., in the cell of the organism in which the nucleic acid occurs), e.g., other chromosomal and extrachromosomal DNA and RNA. The term “isolated” thus encompasses nucleic acids purified by standard nucleic acid purification methods. The term also embraces nucleic acids prepared by recombinant expression in a host cell, as well as chemically synthesized nucleic acids. An “isolated” polypeptide has been substantially separated or purified away from other polypeptides (e.g., in the cell of the organism in which the nucleic acid occurs), e.g., other polypeptides. The term “isolated” thus encompasses polypeptides purified by standard protein purification methods. The term also embraces polypeptides prepared by recombinant expression in a host cell, as well as chemically synthesized polypeptides.

Linker: A linker is an amino acid sequence that covalently links two polypeptide domains. Linkers may be included in the recombinant MHC polypeptides of the present disclosure to provide rotational freedom to the linked polypeptide domains and thereby to promote proper domain folding and inter- and intra-domain bonding. By way of example, in a recombinant polypeptide comprising β1α1, a linker may be provided between the β1 and α1 domains. Linker sequences, which are well known in the art include, but are not limited to, the glycine(4)-serine spacer described by Chaudhary et al. (Nature 339:394-367, 1989)

Recombinant MHC class I α1α2 polypeptides according to the present disclosure include a covalent linkage joining the carboxyl terminus of the α1 domain to the amino terminus of the α2 domain. The α1 and α2 domains of native MHC class I α chains are typically covalently linked in this orientation by an amino acid linker sequence. This native linker may be maintained in the recombinant constructs; alternatively, a recombinant linker may be introduced between the α1 and α2 domains (either in place of or in addition to the native linker sequence).

Nucleic acid molecule: A deoxyribonucleotide or ribonucleotide polymer including, without limitation, cDNA, mRNA, genomic DNA, and synthetic (such as chemically synthesized) DNA. The nucleic acid molecule can be double-stranded or single-stranded. Where single-stranded, the nucleic acid molecule can be the sense strand or the antisense strand.

Pharmaceutical agent or drug: A chemical compound or composition capable of inducing a desired therapeutic or prophylactic effect when properly administered to a subject.

Pharmaceutically acceptable carriers: The pharmaceutically acceptable carriers useful with the polypeptides and nucleic acids described herein are conventional. Remington: The Science and Practice of Pharmacy, The University of the Sciences in Philadelphia, Editor, Lippincott, Williams, & Wilkins, Philadelphia, Pa., 21^{st} Edition (2005), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide or Protein: A polymer in which the monomers are amino acid residues which are joined together through amide bonds. When the amino acids are alpha-amino acids, either the L-optical isomer or the D-optical isomer can be used. The terms “polypeptide,” “peptide,” or “protein” as used herein are intended to encompass any amino acid sequence and include modified sequences such as glycoproteins. The term “polypeptide” or “protein” is specifically intended to cover naturally occurring proteins, as well as those which are recombinantly or synthetically produced.

Purified: The term purified does not require absolute purity; rather, it is intended as a relative term. Thus, for example, a purified recombinant MHC polypeptide preparation is one in which the recombinant MHC polypeptide is more pure than the polypeptide in its originating environment within a cell or preparation. A preparation of a recombinant MHC polypeptide is typically purified such that the recombinant MHC polypeptide represents at least 50% of the total protein content of the preparation. However, more highly purified preparations may be required for certain applications. For example, for such applications, preparations in which the MHC polypeptide comprises at least 75%, at least 90%, at least 95%, at least 98%, at least 99%, or more of the total protein content may be employed.

Recombinant: A recombinant nucleic acid or polypeptide is one that has a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Variants of MHC domain polypeptides will possess a relatively high degree of sequence identity when aligned using standard methods.

Methods of alignment of sequences for comparison are well known in the art. Altschul et al. (1994) presents a detailed consideration of sequence alignment methods and homology calculations. The NCBI Basic Local Alignment Search Tool (BLAST) (Altschul et al., 1990) is available from several sources, including the National Center for Biotechnology Information (ncbi.nlm.nih.gov), for use in connection with the sequence analysis programs blastp, blastn, blastx, tblastn and tblastx. A description of how to determine sequence identity using this program is available at the NCBI website, as are the default parameters.

Variants of MHC domain polypeptides are typically characterized by possession of at least 50% sequence identity counted over the full length alignment with the amino acid sequence of a native MHC domain polypeptide using the NCBI Blast 2.0, gapped blastp set to default parameters. Proteins with even greater similarity to the reference sequences will show increasing percentage identities when assessed by this method, such as at least 60%, at least 65%, at least 70%, at least 75%, at least 80%, at least 90% or at least 95% sequence identity. When less than the entire sequence is being compared for sequence identity, variants will typically possess at least 75% sequence identity over short windows of 10-20 amino acids, and may possess sequence identities of at least 85% or at least 90% or 95% depending on their similarity to the reference sequence. Methods for determining sequence identity over such short windows are described at the NCBI website. Variants of MHC domain polypeptides also retain the biological activity of the native polypeptide. For the purposes of this disclosure, that activity is conveniently assessed by incorporating the variant domain in the appropriate β1α1 or α1α2 polypeptide and determining the ability of the resulting polypeptide to inhibit antigen specific T-cell proliferation in vitro, or to induce T suppressor cells or the expression of IL-10.

Subject: Living multi-cellular vertebrate organisms, a category that includes both human and non-human mammals. Subjects include veterinary subjects, including livestock such as cows and sheep, rodents (such as mice and rats), and non-human primates.

Tolerance: Diminished or absent capacity to make a specific immune response to an antigen. Tolerance is often produced as a result of contact with an antigen in the presence of a two domain MHC molecule, as described herein. In one embodiment, a B cell response is reduced or does not occur. In another embodiment, a T cell response is reduced or does not occur. Alternatively, both a T cell and a B cell response can be reduced or not occur.

Unless otherwise defined, all technical and scientific terms used herein have the same meaning as commonly understood by one of ordinary skill in the art to which the subject matter disclosed herein belongs. The singular terms “a”, “an”, and “the” include plural referents unless context clearly indicates otherwise. Similarly, the word “or” is intended to include “and” unless the context clearly indicates otherwise. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### II. Covalently Linked Recombinant MHC Polypeptide and Antigenic Determinant

Disclosed herein are compositions that include a recombinant MHC polypeptide (such as a purified recombinant MHC polypeptide) covalently linked to an antigenic determinant (such as a purified antigenic determinant) by a disulfide bond. The MHC polypeptide includes covalently linked first and second domains. In some embodiments, the first domain is a mammalian MHC class II β1 domain and the second domain is a mammalian MHC class II α1 domain, wherein the amino terminus of the α1 domain is covalently linked to the carboxyl terminus of the 131 domain and the MHC class II molecule does not include an α2 or P2 domain. In other embodiments, the first domain is a mammalian MHC class I α1 domain and the second domain is a mammalian MHC class I α2 domain, wherein the amino terminus of the α2 domain is covalently linked to the carboxyl terminus of the al domain and the MHC class I molecule does not include an α3 domain. In some examples, the MHC domains are human MHC domains. Two domain MHC polypeptides are described in more detail below and in U.S. Pat. Nos. 6,270,772; 6,815,171; and 7,265,218 and U.S. Pat. Publication Nos. 2008/0267987 and 2005/0142142; each of which are herein incorporated by reference in their entirety.

The recombinant MHC polypeptide is covalently linked to an antigenic determinant, such as a peptide antigen, through a disulfide linkage. The covalent linkage between the recombinant MHC polypeptide and the peptide antigen is formed, for example, by contacting the recombinant MHC polypeptide and the peptide antigen under appropriate conditions for formation of a disulfide linkage. Such conditions can be determined by one of skill in the art utilizing routine methods. Exemplary methods are discussed in further detail below (Section V).

Recombinant MHC polypeptides of the disclosure can be readily produced by expression of a nucleic acid encoding the MHC polypeptide (such as a β1α1 polypeptide or an α1α2 polypeptide) in prokaryotic or eukaryotic cells and purified in large quantities. In some embodiments, the disclosed compositions are produced by contacting a purified recombinant MHC polypeptide (such as a β1α1 polypeptide or an α1α2 polypeptide) and an antigenic determinant (such as a peptide antigen) under conditions sufficient for formation of a disulfide bond between the antigenic determinant and the recombinant MHC polypeptide.

In some examples, the disulfide linkage is formed utilizing a naturally occurring cysteine residue in the MHC polypeptide (such as a cysteine residue in the MHC class II β1 domain or a cysteine residue in the MHC class I α1 or α2 domain). In some examples, the disulfide linkage includes Cys 17 of a recombinant MHC class II β1α1 polypeptide. In other examples, the disulfide linkage includes Cys 79 of a recombinant MHC class II β1α1 polypeptide. In other examples, the disulfide linkage includes Cys 15 and/or Cys 79 of a recombinant MHC class II β1α1 polypeptide. In particular examples, the disulfide linkage includes Cys 17 and/or Cys 79 of a disclosed recombinant MHC class II DR β1α1 polypeptide (for example, Cys 17 and/or Cys 79 of a DR2 MHC polypeptide, such as SEQ ID NO: 4). In other examples, the disulfide linkage includes Cys 16 and/or Cys 78 of a disclosed recombinant MHC class II DP β1α1 polypeptide (for example Cys 16 and/or Cys 78 of a DP2 MHC polypeptide, such as SEQ ID NO: 19). In still further examples, the disulfide linkage includes Cys 16 and/or Cys 80 of a disclosed recombinant MHC class II DQ β1α1 polypeptide (for example, Cys 16 and/or Cys 80 of a DQ2 MHC polypeptide, for example SEQ ID NO: 20). In other examples, the disulfide linkage includes Cys 15, Cys 16, Cys 17, Cys 18, Cys 19, Cys 20, Cys 21, Cys 76, Cys 77, Cys 78, Cys 79, Cys 80, Cys 81, and/or Cys 82 of a β1α1 polypeptide.

In other examples, the disulfide linkage includes Cys 101 of a recombinant MHC class I α1α2 polypeptide. In other examples, the disulfide linkage includes Cys 164 of a recombinant MHC class I α1α2 polypeptide. In other examples, the disulfide linkage includes Cys 98, Cys 99, Cys 100, Cys 102, Cys 103, Cys 104, Cys 161 Cys 162, Cys 163, Cys 165, Cys 166, and/or Cys 167 of an α1α2 polypeptide.

In further examples, the disulfide linkage is also formed utilizing a naturally occurring cysteine residue in a peptide antigen. A naturally occurring cysteine residue includes a cysteine residue that occurs in the native or wild type sequence of a polypeptide (such as a recombinant MHC polypeptide or domain or a peptide antigen).

In other examples, the disulfide linkage is formed utilizing a non-naturally occurring cysteine residue in a recombinant MHC polypeptide, such as a cysteine residue introduced in the MHC polypeptide by mutagenesis. In further examples, the disulfide linkage is formed utilizing a non-naturally occurring cysteine residue in the peptide antigen, such as a cysteine residue introduced in the peptide antigen by mutagenesis. A non-naturally occurring cysteine residue includes a cysteine residue in a polypeptide (such as a recombinant MHC polypeptide or domain or a peptide antigen) that does not occur in the native or wild type polypeptide. In some examples, a non-naturally occurring cysteine residue includes a cysteine residue that replaces any other naturally occurring amino acid in the polypeptide. In other examples, a non-naturally occurring cysteine residue includes a cysteine residue that is added to or inserted in the polypeptide (for example, added at the 5′ or 3′ end of the polypeptide or inserted between two naturally occurring residues in the polypeptide). Any combination of naturally occurring and non-naturally occurring cysteine residues can be utilized for formation of the disulfide bond. In one examples, a non-naturally occurring cysteine is introduced by replacing an amino acid residue in an MHC II α1 domain, for example amino acid position 62 or 72 of the native α1 chain (such as amino acid positions 158 or 168 of a DR, DP, or DQ β1α1 polypeptide, for example SEQ ID NOs: 11, 19, or 20).

Methods of introducing a non-naturally occurring residue in a polypeptide are known to one of skill in the art, and include site-directed mutagenesis of a nucleic acid molecule encoding the polypeptide. See, e.g., Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. One of skill in the art can identify an appropriate residue to mutate to a cysteine residue in a recombinant MHC polypeptide or peptide antigen, for example utilizing molecular modeling and/or constructing and testing constructs for formation of complexes (for example by utilizing the methods described in the Examples below).

A. Recombinant MHC Class II β1α1 Molecules

The amino acid sequences of mammalian MHC class II α and β chain proteins, as well as nucleic acids encoding these proteins, are well known in the art and available from numerous sources including GenBank (ncbi.nlm.nih.gov). Exemplary sequences are provided in Auffray et al., Nature 308:327-333, 1984 (human HLA DQ α); Larhammar et al., Proc. Natl. Acad. Sci. USA 80:7313-7317, 1983 (human HLA DQ β); Das et al., Proc. Natl. Acad. Sci. USA 80:3543-3547, 1983 (human HLA DR α); Tonnell et al., EMBO J. 4:2839-2847, 1985 (human HLA DR β); Lawrence et al., Nucl. Acids Res. 13:7515-7528, 1985 (human HLA DP α); Kelly et al., Nucl. Acids Res. 13:1607-1621, 1985 (human HLA DP β); Syha et al., Nucl. Acids Res. 17:3985, 1989 (rat RT1.B cc); Syha-Jedelhauser et al., Biochim. Biophys. Acta 1089:414-416, 1991 (rat RT1.B β); Benoist et al., Proc. Natl. Acad. Sci. USA 80:534-538, 1983 (mouse I-A α); Estess et al., Proc. Natl. Acad. Sci. USA 83:3594-3598, 1986 (mouse I-A β), all of which are incorporated by reference herein. In one embodiment, the MHC class II protein is a human MHC class II protein.

The recombinant MHC class II molecules of the present disclosure comprise the β1 domain of the MHC class II β chain covalently linked to the α1 domain of the MHC class II α chain. The α1 and β1 domains are well defined in mammalian MHC class II proteins. Typically, the α1 domain is regarded as comprising about residues 1-90 of the mature chain. The native peptide linker region between the α1 and α2 domains of the MHC class II protein spans from about amino acid 76 to about amino acid 93 of the α chain, depending on the particular α chain under consideration. Thus, an α1 domain may include about amino acid residues 1-90 of the α chain, but one of skill in the art will recognize that the C-terminal cut-off of this domain is not necessarily precisely defined, and, for example, might occur at any point between amino acid residues 70-100 of the α chain. In non-limiting examples, the α1 domain includes amino acid residues 1-80, 1-81, 1-82, 1-83, 1-84, 1-85, 1-86, 1-87, 1-88, 1-89, 1-90, 1-91, 1-92, or 1-93 of the α chain. The composition of the α1 domain may also vary outside of these parameters depending on the mammalian species and the particular α chain in question. One of skill in the art will appreciate that the precise numerical parameters of the amino acid sequence are much less important than the maintenance of domain function.

Similarly, the β1 domain is typically regarded as comprising about residues 1-90 of the mature β chain. The linker region between the β1 and β2 domains of the MHC class II protein spans from about amino acid 85 to about amino acid 100 of the β chain, depending on the particular β chain under consideration. Thus, the β1 protein may include about amino acid residues 1-100, but one of skill in the art will again recognize that the C-terminal cut-off of this domain is not necessarily precisely defined, and, for example, might occur at any point between amino acid residues 75-105 of the β chain. In non-limiting examples, the β1 domain includes amino acid residues 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, or 1-100 of the β chain. The composition of the β1 domain may also vary outside of these parameters depending on the mammalian species and the particular β chain in question. Again, one of skill in the art will appreciate that the precise numerical parameters of the amino acid sequence are much less important than the maintenance of domain function. In one embodiment, the β1α1 molecules do not include a β2 domain. In another embodiment, the β1α1 molecules do not include an α2 domain. In yet a further embodiment, the β1α1 molecules do not include either an α2 or a β2 domain.

In certain embodiments, a peptide linker is provided between the β1 and α1 domains. Typically, this linker is at least 6 amino acids in length (for example at least 10, at least 15, at least 20, at least 25, at least 50, or more amino acids), and serves to provide flexibility between the domains such that each domain is free to fold into its native conformation. In particular examples, the linker is about 2 to 25 amino acids in length (for example, 6 to 25 or 15 to 20 amino acids). One of skill in the art can select an appropriate linker, if included in the recombinant MHC polypeptide. The linker sequence may conveniently be provided by designing the PCR primers to encode the linker sequence.

B. Recombinant MHC Class I α1α2 Molecules

The amino acid sequences of mammalian MHC class I α chain proteins, as well as nucleic acids encoding these proteins, are well known in the art and available from numerous sources including GenBank (ncbi.nlm.nih.gov). Exemplary sequences are provided in Browning et al., Tissue Antigens 45:177-187, 1995 (human HLA-A); Kato et al., Immunogenet. 37:212-216, 1993 (human HLA-B); Steinle et al., Tissue Antigens 39:134-134, 1992 (human HLA-C); Walter et al., Immunogenetics 41:232, 1995 (rat Ia); Walter et al., Immunogenetics 39:351-354, 1994 (rat Ib); Kress et al., Nature 306:602-604, 1983 (mouse H-2-K); Schepart et al., J. Immunol. 136:3489-3495, 1986 (mouse H-2-D); and Moore et al., Science 215:679-682, 1982 (mouse H-2-1), which are incorporated by reference herein. In one embodiment, the MHC class I protein is a human MHC class I protein.

The recombinant MHC class I molecules of the present disclosure comprise the α1 domain of the MHC class I α chain covalently linked to the α2 domain of the MHC class I chain. These two domains are well defined in mammalian MHC class I proteins. Typically, the α1 domain is regarded as comprising about residues 1-90 of the mature chain and the α2 chain as comprising about amino acid residues 90-180, although the cut-off points are not precisely defined and will vary between different MHC class I molecules. In non-limiting examples, the α1 domain includes amino acid residues 1-80, 1-81, 1-82, 1-83, 1-84, 1-85, 1-86, 1-87, 1-88, 1-89, 1-90, 1-91, 1-92, or 1-93 of the α chain. The boundary between the α2 and α3 domains of the MHC class I α protein typically occurs in the region of amino acids 179-183 of the mature chain. In non-limiting examples, the α2 domain includes amino acid residues 85-180, 86-180, 87-180, 88-180, 89-180, 90-180, 90-179, 90-181, 90-182, or 90-183, of the α chain. The composition of the α1 and α2 domains may also vary outside of these parameters depending on the mammalian species and the particular α chain in question. One of skill in the art will appreciate that the precise numerical parameters of the amino acid sequence are much less important than the maintenance of domain function. In one embodiment, the α1α2 molecule does not include an α3 domain.

The α1α2 construct may be most conveniently constructed by amplifying the reading frame encoding the dual-domain (α1 and α2) region between amino acid number 1 and amino acids 179-183, although one of skill in the art will appreciate that some variation in these end-points is possible. Such a molecule includes the native linker region between the α1 and α2 domains, but if desired that linker region may be removed and replaced with a synthetic linker peptide (such as a linker described above).

C. Modified MHC Molecules

While the foregoing discussion uses as examples naturally occurring MHC class I and class II molecules and the various domains of these molecules, one of skill in the art will appreciate that variants of these molecules and domains may be made and utilized in the same manner as described. Thus, reference herein to a domain of an MHC polypeptide or molecule (e.g., an MHC class II β1 domain) includes both naturally occurring forms of the referenced molecule, as well as molecules that are based on the amino acid sequence of the naturally occurring form, but which include one or more amino acid sequence variations. Such variant polypeptides may also be defined in the degree of amino acid sequence identity that they share with the naturally occurring molecule. Typically, MHC domain variants will share at least 80% sequence identity with the sequence of the naturally occurring MHC domain. More highly conserved variants will share at least 90% or at least 95% sequence identity with the naturally occurring sequence. Variants of MHC domain polypeptides also retain the biological activity of the naturally occurring polypeptide. For the purposes of this disclosure, that activity is conveniently assessed by incorporating the variant domain in the appropriate β1α1 or α1α2 polypeptide and determining the ability of the resulting polypeptide to inhibit antigen specific T-cell proliferation in vitro. Methods of determining antigen-specific T-cell proliferation are well known to one of skill in the art (see, e.g., Huan et al., J. Chem. Technol. Biotechnol. 80:2-12, 2005).

Variant MHC domain polypeptides include proteins that differ in amino acid sequence from the naturally occurring MHC polypeptide sequence but which retain the specified biological activity. Such proteins may be produced by manipulating the nucleotide sequence of the molecule encoding the domain, for example by site-directed mutagenesis or the polymerase chain reaction. The simplest modifications involve the substitution of one or more amino acids for amino acids having similar biochemical properties. These so-called conservative substitutions are likely to have minimal impact on the activity of the resultant protein.

In some embodiments, the disclosed recombinant MHC polypeptides include modified MHC polypeptides that include one or more amino acid changes that decrease self-aggregation of native MHC polypeptides or β1α1 or α1α2 MHC polypeptides. See, e.g., U.S. Pat. Publ. No. 2005/0142142 and Huan et al., J. Chem. Technol. Biotechnol. 80:2-12, 2005; both of which are incorporated herein by reference. Modified MHC polypeptides of the disclosure are rationally designed and constructed to introduce one or more amino acid changes at a solvent-exposed target site located within, or defining, a self-binding interface found in the native MHC polypeptide. The self-binding interface that is altered in the modified MHC polypeptides typically includes one or more amino acid residues that mediate self-aggregation of a native MHC polypeptide, or of an “unmodified” β1α1 or α1α2 MHC polypeptide incorporating the native MHC polypeptide. Although the self-binding interface is correlated with the primary structure of the native MHC polypeptide, this interface may only appear as an aggregation-promoting surface feature when the native polypeptide is isolated from the intact MHC complex and incorporated in the context of an “unmodified” β1α1 or α1α2 MHC molecule. In the case of exemplary MHC class II molecules described herein (e.g., comprising linked β1 and α1 domains), the native β1α1 structure only exhibits certain solvent-exposed, self-binding residues or motifs after removal of Ig-fold like β2 and α2 domains found in the intact MHC II complex. These same residues or motifs that mediate aggregation of unmodified β1α1 MHC molecules, are presumptively “buried” in a solvent-inaccessible conformation or otherwise “masked” (e.g., prevented from mediating self-association) in the native or progenitor MHC II complex (likely through association with the Ig-fold like β2 and α2 domains).

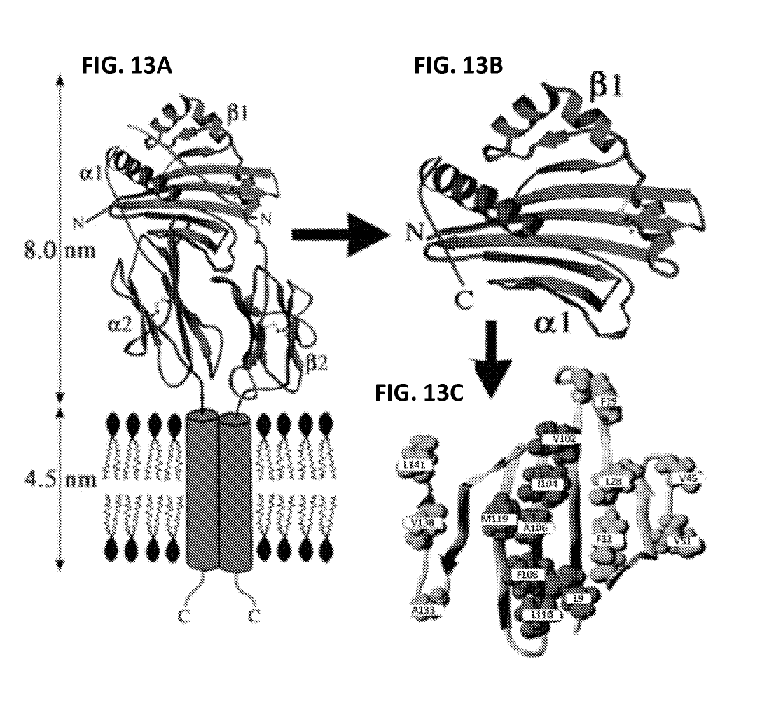

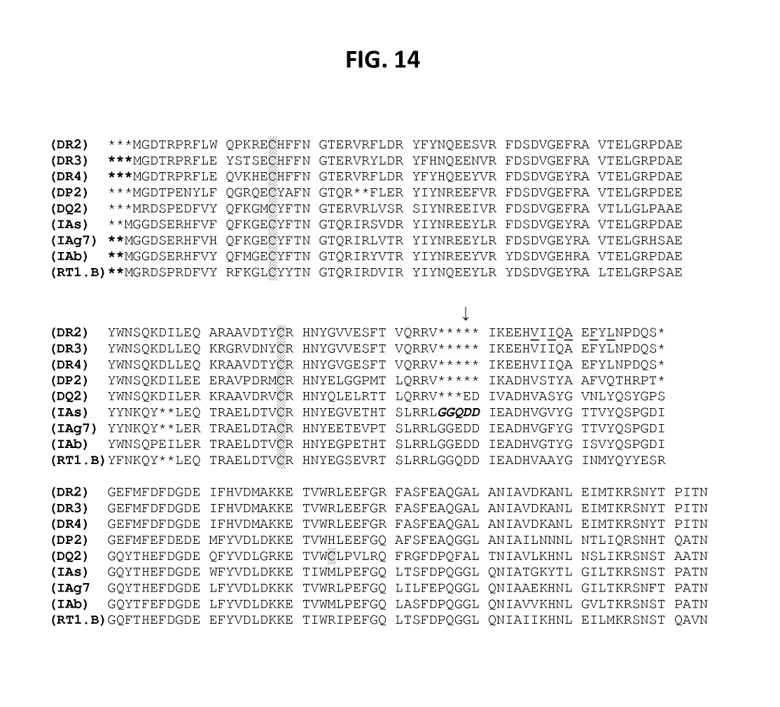

In some examples, surface modification of an MHC molecule comprising an MHC class II component to yield much less aggregation prone form can be achieved, for example, by replacement of one or more hydrophobic residues identified in the β-sheet platform of the MHC component with non-hydrophobic residues, for example polar or charged residues. FIGS. 13A-C depict an exemplary HLA-DR2 polypeptide, an exemplary β1α1 molecule, and hydrophobic β-sheet platform residues that may be targeted for modification, respectively. In some examples, one or more hydrophobic amino acids of a central core portion of the β-sheet platform are modified, such as one or more of V102, I104, A106, F108, and L110 of a human DR2 MHC class II β1α1 RTL (for example, SEQ ID NO: 11). In other examples, one or more hydrophobic amino acids of a central core portion of the β-sheet platform are modified, such as one or more of V98, A102, and F104 of a human DP2 MHC class II β1α1 RTL (for example, SEQ ID NO: 19). In further examples, one or more hydrophobic amino acids of a central core portion of the β-sheet platform are modified, such as one or more of V104, and G108 of a human DQ2 MHC class II β1α1 RTL (for example, SEQ ID NO: 20). One of skill in the art can identify corresponding amino acids in other MHC class II molecules or β1α1 molecules. See, e.g., the alignment of human, mouse, and rat RTLs provided in FIG. 14.

In particular examples, one or more of the identified hydrophobic β-sheet platform amino acids is changed to either to a polar (for example, serine) or charged (for example, aspartic acid) residue. In some examples all five of V102, I104, A106, F108, and L110 (of SEQ ID NO: 11 or corresponding amino acids in another β1α1 polypeptide) are changed to a polar or charged residue. In one example, each of V102, I104, A106, F108, and L110 of SEQ ID NO: 11 (or corresponding amino acids in other β1α1 polypeptides) are changed to an aspartic acid residue.

In other examples, additional hydrophobic target residues are available for modification to alter self-binding characteristics of the β-sheet platform portion of class II MHC molecules incorporated in MHC molecules. In reference to FIG. 13C, the left arm of the diagrammed β-sheet platform includes a separate “motif” of three noted hydrophobic residues (top to bottom), L141, V138, and A133 of SEQ ID NO: 11 (or corresponding amino acids in other β1α1 polypeptides) that can be modified to a non-hydrophobic (e.g., polar, or charged) residue. In some examples, L141, V138, and A133 of SEQ ID NO: 11 correspond to L139, V136, and Dβ1 of SEQ ID NO: 19 or V141, K138, and V133 of SEQ ID NO: 20. Also in reference to FIG. 13C, several target hydrophobic residues are marked to the right of the core β-sheet motif, including L9, F19, L28, F32, V45, and V51 of SEQ ID NO: 11 (or corresponding amino acids in other β1α1 polypeptides), which may be regarded as one or more additional, self-binding or self-associating target “motifs” for MHC molecule modification. In some examples, L9, F19, L28, F32, V45, and V51 of SEQ ID NO: 11 correspond to L9, F19, L26, I30, V43, and V49 of SEQ ID NO: 19 or V9, T19, V28, I32, V45, and V51 of SEQ ID NO: 20. One of skill in the art can identify corresponding amino acids in other MHC class II molecules or 662 1α1 molecules. Any one or a combination of these residues may be targeted for modification to a non-hydrophobic residue, increasing monomeric MHC molecules.

The modified MHC molecules disclosed herein yield an increased percentage of monodisperse (monomeric) molecules in solution compared to a corresponding, unmodified MHC molecule (e.g., comprising the native MHC polypeptide and bearing the unmodified, self-binding interface). In certain embodiments, the percentage of unmodified MHC molecule present as a monodisperse species in aqueous solution may be as low as 1%, more typically 5-10% or less of total MHC protein, with the balance of the unmodified MHC molecule being found in the form of higher-order aggregates. In contrast, modified MHC molecules disclosed herein yield at least 10%-20% monodisperse species in solution. In other embodiments, the percentage of monomeric species in solution will range from 25%-40%, often 50%-75%, up to 85%, 90%, 95%, or greater of the total MHC protein present, with a commensurate reduction in the percentage of aggregate MHC species compared to quantities observed for the corresponding, unmodified MHC molecules under comparable conditions.

D. Antigenic Determinants

The disclosed compositions include an antigenic determinant (such as a peptide antigen) covalently linked to a recombinant MHC polypeptide, such as those described above. Any antigenic peptide that is conventionally associated with class I or class II MHC molecules and recognized by a T-cell can be used for this purpose. Antigenic peptides from a number of sources have been characterized in detail, including antigenic peptides from honey bee venom allergens, dust mite allergens, toxins produced by bacteria (such as tetanus toxin) and human tissue antigens involved in autoimmune diseases. Detailed discussions of such peptides are presented in U.S. Pat. Nos. 5,595,881; 5,468,481; and 5,284,935; each of which is incorporated herein by reference.

As is well known in the art (see for example U.S. Pat. No. 5,468,481) the presentation of antigen in MHC complexes on the surface of APCs generally does not involve a whole antigenic peptide. Rather, a peptide located in the groove between the β1 and α1 domains (in the case of MHC II) or the α1 and α2 domains (in the case of MHC I) is typically a small fragment of the whole antigenic peptide. As discussed in Janeway & Travers (Immunobiology: The Immune System in Health and Disease, Current Biology Ltd., New York, 1997), peptides located in the peptide groove of MHC class I molecules are constrained by the size of the binding pocket and are typically 8-15 amino acids long, more typically 8-10 amino acids in length (but see Collins et al., Nature 371:626-629, 1994 for possible exceptions). In contrast, peptides located in the peptide groove of MHC class II molecules are not constrained in this way and are often much larger, typically at least 11 amino acids in length (such as about 13-25 amino acids). Peptide fragments for loading into MHC molecules can be prepared by standard means, such as use of synthetic peptide synthesis machines.

Exemplary antigenic determinants include peptides identified in the pathogenesis of autoimmune disease. In some examples, the antigenic determinant is a peptide identified in the pathogenesis of rheumatoid arthritis (e.g., type II collagen), myasthenia gravis (acetyl choline receptor), multiple sclerosis (MBP, PLP, or MOG), uveitis or other retinal diseases (interphotoreceptor retinoid binding protein (IRBP), arrestin, recoverin, and phosducin), and diabetes (insulin).

Particular antigenic determinants include but are not limited to MOG peptides, such as MOG 35-55 (SEQ ID NO: 5), MOG 1-25 (GQFRVIGPRHPIRALVGDEVELPCR; SEQ ID NO: 21), MOG 94-116 (GGFTCFFRDHSYQEEAAMELKVE; SEQ ID NO: 22), MOG 145-160 (VFLCLQYRLRGKLRAE; SEQ ID NO: 23), or MOG 194-208 (LVALIICYNWLHRRL; SEQ ID NO: 24); MBP peptides, such as MBP 10-30 (RHGSKYLATASTMDHARHGFL; SEQ ID NO 25), MBP 35-45 (DTGILDSIGRF; SEQ ID NO: 26), MBP 77-91 (SHGRTQDENPVVHF; SEQ ID NO: 27), MBP 85-99 (ENPVVHFFKNIVTPR; SEQ ID NO: 28), MBP 95-112 (IVTPRTPPPSQGKGRGLS; SEQ ID NO: 29), or MBP 145-164 (VDAQGTLSKIFKLGGRDSRS; SEQ ID NO: 30); and PLP peptides, such as PLP 139-151 (CHCLGKWLGHPDKFVG; SEQ ID NO: 16), or PLP 95-116 (GAVRQIFGDYKTTICGKGLSAT; SEQ ID NO: 31). Additional exemplary antigenic determinants include, but are not limited to, collagen type II peptides, such as collagen 11261-274 (AGFKGEQGPKGEPG; SEQ ID NO: 32), collagen II 259-273 (GIAGFKGEQGPKGEP; SEQ ID NO: 33), collagen II 257-270 (EPGIAGFKGEQGPK; SEQ ID NO: 34), or modified collagen II 257-270 (APGIAGFKAEQAAK; SEQ ID NO: 35).

Further exemplary antigenic determinants include IRBP peptides, such as IRBP 1177-1191 (ADGSSWEGVGVVPDV; SEQ ID NO: 36); arrestin peptides, such as arrestin 291-310 (NRERRGIALDGKIKHEDTNL; SEQ ID NO: 37); phosducin peptides, such as phosducin 65-96 (KERMSRKMSIQEYELIHQDKEDEGCLRKYRRQ; SEQ ID NO: 38); or recoverin peptides, such as recoverin 48-52 (QFQSI; SEQ ID NO: 39), recoverin 64-70 (KAYAQHV; SEQ ID NO: 40), recoverin 62-81 (PKAYAQHVFRSFDANSDGTL; SEQ ID NO: 41), or recoverin 149-162 (EKRAEKIWASFGKK; SEQ ID NO: 42). Additional exemplary antigenic determinants include fibrinogen-α peptides, such as fibrinogen-α 40-59 (VERHQSACKDSDWPFCSDED; SEQ ID NO: 43), fibrinogen-α 616-625 (THSTKRGHAKSRPVRGIHTS; SEQ ID NO: 44), fibrinogen-α 79-91 (QDFTNRINKLKNS; SEQ ID NO: 45), or fibrinogen-α 121-140 (NNRDNTYNRVSEDLRSRIEV; SEQ ID NO: 46); vimentin peptides, such as vimentin 59-79 (GVYATRSSAVRLRSSVPGVRL; SEQ ID NO: 47), vimentin 26-44 (SSRSYVTTSTRTYSLGSAL; SEQ ID NO: 48), vimentin 256-275 (IDVDVSKPDLTAALRDVRQQ; SEQ ID NO: 49), or vimentin 415-433 (LPNFSSLNLRETNLDSLPL; SEQ ID NO: 50); α-enolase peptides, such as α-enolase 5-21 (KIHAREIFDSRGNPTVE; SEQ ID NO: 51); or human cartilage glycoprotein 39 peptides, such as human cartilage glycoprotein 39 259-271 (PTFGRSFTLASSE; SEQ ID NO: 52).

In other examples, antigenic determinants include α2-gliadin peptides, such as α2-gliadin 61-71 (FPQPELPYPQP; SEQ ID NO: 53) or α2-gliadin 58-77 (LQPFPQPQLPYPQPQLPYPQ; SEQ ID NO: 54).

In some examples, the antigenic determinant also includes a modification, such as glycosylation or citrullination. In other examples, the antigenic determinant includes one or more additional amino acid substitutions.

In some embodiments, the antigenic determinant (such as a peptide antigen) includes a cysteine residue at the p4 position. In some embodiments, the antigenic determinant includes a cysteine residue that can occupy the P4 pocket of a MHC polypeptide (such as an MHC class II β1α1 polypeptide). The cysteine residue may be a naturally occurring (native) cysteine residue in the antigenic determinant, or may be a non-naturally occurring cysteine residue (for example, introduced by mutagenesis or peptide synthesis). One of skill in the art can identify the p4 position of an antigenic determinant (see, e.g., Corper et al., Science 288:505-511, 2000; Latek et al., Immunity 12:699-710, 2000). For example, MHC class II alleles have peptide binding preferences characterized by a core binding motif for peptides, with anchor residues of the peptide binding into characteristic pockets of each allele. The peptide residues that bind into the pockets are considered “anchor residues.” The P4 pocket is situated close to the native disulfide bond in MHC class II molecules. Therefore, a peptide antigen with a cysteine that occupies the P4 pocket is able to disrupt the native disulfide bond and form a disulfide bond with the MHC polypeptide. Resources for identifying the motifs of all MHC molecules are available (for example, on the world wide web at syfpeithi.de). See also Example 6, below. Cysteine residues can be introduced into an antigenic determinant, for example by mutagenesis, and tested for their ability to form a disulfide bond with a recombinant MHC polypeptide utilizing the methods disclosed herein.

In some examples, a naturally occurring antigenic determinant does not include a cysteine residue and the antigenic determinant is modified to introduce a non-naturally occurring cysteine residue. In some examples, an antigenic determinant is modified to include one or more cysteine residues (for example, to introduce a non-naturally occurring cysteine residue at the p4 position) or to remove one or more cysteine residues, for example, by mutagenesis or peptide synthesis. In some examples, the non-naturally occurring cysteine is present at the amino-terminus or the carboxy-terminus of the antigenic determinant. In other examples, the non-naturally occurring cysteine is present in the amino-terminal half of the antigenic determinant, the amino-terminal third of the antigenic determinant, or the amino-terminal quarter of the antigenic determinant. In further examples, the non-naturally occurring cysteine is present in the carboxy-terminal half of the antigenic determinant, the carboxy-terminal third of the antigenic determinant, or the carboxy-terminal quarter of the antigenic determinant.

In some examples, the peptide antigen includes an insulin peptide, such as insulin B:9-23 (e.g., SEQ ID NO: 3). This peptide includes a naturally occurring cysteine residue at position 19 (position 11 of SEQ ID NO: 3), which in some examples can form a disulfide bond with a cysteine of an MHC polypeptide. In another example, the peptide antigen includes a MOG peptide, such as MOG 35-55 (e.g., SEQ ID NOs: 5 or 6). This peptide does not include a naturally occurring cysteine residue. In some examples, one residue of MOG 35-55 is mutated to a cysteine residue (e.g., any one of SEQ ID NOs: 12, 13, and 15) which can form a disulfide bond with a cysteine of an MHC polypeptide. In a further example, the peptide is a PLP peptide, such as PLP 139-151 (e.g., SEQ ID NO: 16).

### III. Pharmaceutical Formulations and Methods of Treating or Inhibiting an Autoimmune Disorder

The disclosed compositions including a MHC polypeptide covalently linked to a peptide antigen can be used to treat or inhibit conditions mediated by antigen-specific T-cells. Such conditions include allergies, transplant rejection and autoimmune diseases (including but not limited to multiple sclerosis, type I diabetes, rheumatoid arthritis, celiac disease, psoriasis, lupus, uveitis, optic neuritis, pernicious anemia, myasthenia gravis, and Addison's disease). The disclosed compositions can also be used to treat or inhibit other conditions including cognitive and/or neuropsychiatric impairment (such as that induced by substance addiction), retinal disorders (such as a retinal degeneration, a maculopathy, a retinopathy, retinal detachment, or glaucoma). Various forms of MHC polypeptides that may be used to treat these conditions have been previously described and the methods used in those systems are equally useful with the compositions of the present disclosure. Exemplary methodologies are described in U.S. Pat. Nos. 5,130,297, 5,284,935, 5,468,481, 5,734,023 and 5,194,425 (herein incorporated by reference). See also U.S. Provisional Applications 61/437,316, filed Jan. 28, 2011; 61/438,004, filed Jan. 31, 2011; and 61/516,918, filed Apr. 7, 2011; U.S. patent application Ser. No. 12/661,038, filed Mar. 8, 2010; U.S. Pat. Publ. No. 2011/000832; and Huan et al., Mucosal Immunol. 4:112-120, 2011, all of which are incorporated herein by reference.

By way of example, the compositions including an effective amount of an MHC polypeptide covalently linked to a peptide antigen may be administered to a subject in order to induce anergy in self-reactive T-cell populations, or these T-cell populations may be treated by administration of MHC/peptide complexes conjugated with a toxic moiety. Alternatively, the MHC/peptide complexes may be administered to a subject to induce T suppressor cells or to modify a cytokine expression profile.

For administration to a subject (such as a human or non-human mammal), a recombinant MHC polypeptide covalently linked to a peptide antigen disclosed herein is generally combined with a pharmaceutically acceptable carrier. In general, the nature of the carrier will depend on the particular mode of administration being employed. The pharmaceutically acceptable carriers and excipients useful in this disclosure are conventional. See, e.g., Remington: The Science and Practice of Pharmacy, The University of the Sciences in Philadelphia, Editor, Lippincott, Williams, & Wilkins, Philadelphia, Pa., 21^{st} Edition (2005). For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, pH buffering agents, and the like, for example sodium acetate or sorbitan monolaurate.

As is known in the art, protein-based pharmaceuticals may be only inefficiently delivered through ingestion. However, pill-based forms of pharmaceutical proteins may alternatively be administered subcutaneously, particularly if formulated in a slow-release composition. Slow-release formulations may be produced by combining the target protein with a biocompatible matrix, such as cholesterol. Another method of administering protein pharmaceuticals is through the use of mini osmotic pumps. As stated above, a biocompatible carrier would also be used in conjunction with this method of delivery. Additional possible methods of delivery include deep lung delivery by inhalation (Edwards et al., Science 276:1868-1871, 1997) and transdermal delivery (Mitragotri et al., Pharm. Res. 13:411-420, 1996).

The pharmaceutical compositions of the present disclosure may be administered by any means that achieve their intended purpose. In some embodiments, an effective amount of a disclosed composition including a recombinant MHC polypeptide covalently linked to an antigenic determinant is administered to a subject with an autoimmune disorder in order to treat or inhibit the autoimmune disorder. Amounts and regimens for the administration of a composition including the selected MHC polypeptide covalently linked to a peptide antigen can be determined by the attending clinician. Effective doses for therapeutic application will vary depending on the nature and severity of the condition to be treated, the particular MHC polypeptide and peptide antigen selected, the age and condition of the patient and other clinical factors. Typically, the dose range will be from about 0.1 μg/kg body weight to about 100 mg/kg body weight. Other suitable ranges include doses of from about 100 μg/kg to 10 mg/kg body weight or about 500 μg/kg to about 5 mg/kg body weight. The dosing schedule may vary from once a week to daily depending on a number of clinical factors, such as the subject's sensitivity to the protein. Examples of dosing schedules are 3 μg/kg administered twice a week, three times a week or daily; a dose of 7 μg/kg twice a week, three times a week or daily; a dose of 10 μg/kg twice a week, three times a week or daily; or a dose of 30 μg/kg twice a week, three times a week or daily. Additional examples of dosing schedules are about 1 mg/kg administered twice a week, three times a week or daily; a dose of about 5 mg/kg twice a week, three times a week or daily; or a dose of about 10 mg/kg twice a week, three times a week or daily.

The pharmaceutical compositions that include one or more of the disclosed MHC molecules covalently linked to an antigenic determinant can be formulated in unit dosage form, suitable for individual administration of precise dosages. In one specific, non-limiting example, a unit dosage can contain from about 1 ng to about 1000 mg of MHC polypeptide covalently linked to an antigenic determinant (such as about 10 ng to 700 mg, about 1 mg to 500 mg, about 5 mg to 250 mg, or about 10 mg to 100 mg, for example, about 70 mg). The amount of active composition administered will be dependent on the subject being treated, the severity of the affliction, and the manner of administration, and is best left to the judgment of the prescribing clinician. Within these bounds, the formulation to be administered will contain a quantity of the active composition in amounts effective to achieve the desired effect in the subject being treated. In some examples, the MHC molecule is administered daily, weekly, bi-weekly, or monthly.

The compounds of this disclosure can be administered to a subject (such as a subject with an autoimmune disorder or at risk of developing an autoimmune disorder) in various manners such as topically, orally, intravenously, intramuscularly, intraperitoneally, intranasally, intradermally, intrathecally, subcutaneously, intraocularly, via inhalation, or via suppository. In one example, the compounds are administered to the subject subcutaneously. In another example, the compounds are administered to the subject intravenously. The particular mode of administration and the dosage regimen will be selected by the attending clinician, taking into account the particulars of the case (e.g., the subject, the disease, the disease state involved, and whether the treatment is prophylactic). Treatment can involve monthly, bi-monthly, weekly, daily or multi-daily doses of composition over a period of a few days to months, or even years.

In some examples, an effective amount (such as a therapeutically effective amount) of a disclosed recombinant MHC polypeptide covalently linked to an antigenic determinant can be the amount of an MHC polypeptide (such as an MHC class II β1α1 polypeptide or an MHC class I α1α2 polypeptide) covalently linked to an antigen necessary to treat or inhibit an autoimmune disorder (such as multiple sclerosis, type I diabetes, rheumatoid arthritis, celiac disease, psoriasis, systemic lupus erythematosus, or optic neuritis) in a subject.

### IV. ADDITIONAL USES OF COVALENTLY LINKED MHC POLYPEPTIDE AND PEPTIDE ANTIGEN

The compositions of the disclosure including a MHC polypeptide covalently linked to a peptide antigen are useful for in vitro and in vivo applications. Indeed, as a result of the biological activities of these polypeptides, they may be used in numerous applications in place of either intact purified MHC molecules, or APCs that express MHC molecules. As discussed in Section III (above), the disclosed compositions are useful for treating or inhibiting an autoimmune disorder in a subject with such a disorder or at risk for such a disorder. Additional applications are described below.

In vitro applications of the disclosed compositions include the detection, quantification and purification of antigen-specific T-cells. Methods for using various forms of MHC-derived complexes for these purposes are well known and are described in, for example, U.S. Pat. Nos. 5,635,363; 5,595,881; and 6,815,171. For such applications, the disclosed compositions including a MHC polypeptide covalently linked to a peptide antigen may be free in solution or may be attached to a solid support such as the surface of a plastic dish, a microtiter plate, a membrane, or beads. Typically, such surfaces are plastic, nylon or nitrocellulose. Compositions including a MHC polypeptide covalently linked to a peptide antigen in free solution are useful for applications such as fluorescence activated sell sorting (FACS). For detection and quantification of antigen-specific T-cells, the polypeptides are preferably labeled with a detectable marker, such as radionuclides (e.g., gamma-emitting sources such as indium-111), paramagnetic isotopes, fluorescent markers (e.g., fluorescein), enzymes (such as alkaline phosphatase), cofactors, chemiluminescent compounds and bioluminescent compounds. The binding of such labels to the MHC polypeptides may be achieved using standard methods (e.g., U.S. Pat. No. 5,734,023; incorporated herein by reference).

The T-cells to be detected, quantified or otherwise manipulated are generally present in a biological sample removed from a subject. The biological sample is typically blood or lymph, but may also include tissue samples such as lymph nodes, tumors, joints etc. It will be appreciated that the precise details of the method used to manipulate the T-cells in the sample will depend on the type of manipulation to be performed and the physical form of both the biological sample and the MHC molecules. In general terms, the composition including a MHC polypeptide covalently linked to a peptide antigen is added to the biological sample, and the mixture is incubated for sufficient time (e.g., from about 5 minutes up to several hours) to allow binding. Detection and quantification of T-cells bound to the composition including a MHC polypeptide covalently linked to a peptide antigen may be performed by a number of methods including, where the MHC/peptide includes a fluorescent label, fluorescence microscopy and FACS. Standard immunoassays such as ELISA and radioimmunoassay may also be used to quantify T-cell-MHC/peptide complexes where the MHC/peptide complexes are bound to a solid support. In some examples, quantification of antigen-specific T-cell populations is useful in monitoring the course of a disease. For example, in a subject with multiple sclerosis, the efficacy of a therapy administered to reduce the number of antigen-reactive T-cells may be monitored using MHC covalently linked to an antigen (such as MBP) to quantify the number of such T-cells present in the subject.

FACS may also be used to separate T-cell-MHC/peptide complexes from the biological sample, which may be particularly useful where a specified population of antigen-specific T-cells is to be removed from the sample, such as for enrichment purposes. Where the MHC/peptide complex is bound to magnetic beads, the binding T-cell population may be purified as described by Miltenyi et al., Cytometry 11:231-238, 1990.

A specified antigen-specific T-cell population in the biological sample may be anergized by incubation of the sample with compositions including an MHC polypeptide covalently linked to a peptide antigen containing the peptide recognized by the targeted T-cells. Thus, when these compositions bind to the T cell receptor in the absence of other co-stimulatory molecules, a state of anergy is induced in the T-cell. Such an approach is useful in situations where the targeted T-cell population recognizes a self-antigen, such as in various autoimmune diseases. Alternatively, the targeted T-cell population may be killed directly by incubation of the biological sample with an MHC/peptide complex conjugated with a toxic moiety.

T-cells may also be activated in an antigen-specific manner by the polypeptides of the disclosure. For example, the disclosed compositions including a MHC polypeptide covalently linked to a peptide antigen may be adhered at a high density to a solid surface, such as a plastic dish or a magnetic bead. Exposure of T-cells to the polypeptides on the solid surface can stimulate and activate T-cells in an antigen-specific manner, despite the absence of co-stimulatory molecules. This is likely attributable to sufficient numbers of T cell receptors on a T-cell binding to the MHC/peptide complexes that co-stimulation is unnecessary for activation.

In one embodiment, suppressor T cells are induced. Thus, when the composition including a MHC polypeptide covalently linked to a peptide antigen binds to the T cell receptor in the proper context, suppressor T cells are induced in vitro. In one embodiment, effector functions are modified, and cytokine profiles are altered by incubation with a MHC/peptide complex.

### V. METHODS AND KITS FOR MAKING A RECOMBINANT MHC POLYPEPTIDE COVALENTLY LINKED TO A PEPTIDE ANTIGEN

In some embodiments, the disclosed compositions are prepared by contacting an antigenic determinant (such as a peptide antigen) with a recombinant MHC polypeptide (such as a α1α polypeptide or an α1α2 polypeptide) under conditions sufficient for formation of a disulfide linkage between the antigenic determinant and the MHC polypeptide. In some embodiments one or more (such as 1, 2, 3, 4, 5, or more) antigenic determinants are incubated with a recombinant MHC polypeptide (such as a β1α polypeptide or an α1α2 polypeptide) under conditions sufficient for formation of a disulfide linkage between an antigenic determinant and the MHC polypeptide, thereby producing a mixed population of MHC polypeptides each linked to a different antigenic determinant. In other examples, a mixed population of MHC polypeptides linked to different antigenic determinants is produced by incubating each antigenic determinant with a recombinant MHC polypeptide under conditions sufficient for formation of a disulfide bond and then mixing together the resulting MHC polypeptides covalently linked to an antigenic determinant. In some examples, the MHC polypeptide is the same in each reaction. In other examples, the MHC polypeptide is different in each reaction, producing a mixed population of MHC polypeptides and/or antigenic determinants.

In some embodiments, conditions sufficient for formation of a disulfide linkage between a recombinant MHC polypeptide and an antigenic determinant include a molar excess of one or more antigenic determinants. In some examples the ratio of antigenic determinant to recombinant MHC polypeptide is at least 1.1:1 (such as at least 1.5:1, 2:1, 3:1, 4:1, 5:1, 6:1, 7:1, 8:1, 9:1, 10:1, 15:1, 20:1, 25:1, 50:1, 50:1, 100:1, or more). In other examples, the ratio of antigenic determinant to recombinant MHC polypeptide is about 1:1 to about 500:1 (such as about 2:1 to 100:1, about 5:1 to 50:1, or about 10:1 to 20:1). In one non-limiting example, the ratio of antigen determinant to recombinant MHC polypeptide is about 10:1. One of skill in the art will appreciate that the disclosed compositions can also be produced by contacting a molar excess of a recombinant MHC polypeptide with an antigenic determinant, for example by adjusting other reaction parameters, such as time and/or temperature of incubation.

In some embodiments, conditions sufficient for formation of a disulfide linkage between an antigenic determinant and a recombinant MHC polypeptide include incubation in a buffer or solution that promotes redox capture of the antigenic determinant at a temperature and for a period of time sufficient for the disulfide bond to form. Exemplary solutions for redox capture include solutions with a pH of about 5 to about 8.5 (for example, about 5.5 to 8.0, about 6 to 7.5, or about 6.5 to 8.5) In some examples, the pH of the solution is about 6.0 to 7.0 (such as about 6.0, 6.1, 6.2, 6.3, 6.4, 6.5, 6.6., 6.7, 6.8, 6.9, or 7.0). In one non-limiting example, the solution has a pH of about 6.5. In some examples, utilizing a lower pH (for example, about 6 to 7) promotes formation of a disulfide bond between the MHC polypeptide and the antigenic determinant and decrease formation of homodimers by the antigenic determinant. This may be particularly advantageous when the MHC polypeptide includes an acidic amino acid residue (for example, aspartic acid or glutamic acid) close to the cysteine residue involved in disulfide bond formation (for example, within about 1-10 Å, such as about 2-5 Å). In some examples, the solution optionally includes a mild reducing agent (such as glutathione, β-mercaptoethanol, or dithiothreitol), which can also decrease homodimer formation by the antigenic determinant.

The solution also includes a detergent (for example, an ionic detergent, a zwitterionic detergent, or a nonionic detergent). Suitable detergents can be selected by one of skill in the art and include sodium dodecyl sulfate (SDS), hexadecyltrimethylammonium bromide (CTAB), 3-[(3-cholamidopropyl)dimethylammonio]-1-propanesulfonate (CHAPS), [(3-cholamidopropyl)dimethylammonio]-2-hydroxy-1-propanesulfonate (CHAPSO), Tween® detergents (such as Tween®-20), Triton® detergents (such as Triton®X100), or Brij® detergents (such as Brij® 35). In some examples, the solution includes about 0.01-0.1% detergent, such as about 0.025-0.075%, or about 0.05% (for example, about 0.01%, 0.015%, 0.02%, 0.025%, 0.03%, 0.035%, 0.04%, 0.045%, 0.05%, 0.055%, 0.06%, 0.065%, 0.07%, 0.075%, 0.08%, 0.085%, 0.09%, 0.095%, or 0.1% detergent). In one non-limiting example, the solution includes about 0.05% SDS. In some embodiments, the solution includes additional components, such as one or more salts (for example, NaCl) and/or a preservative agent (for example, NaN₃). In a particular example, the solution includes 100 mM NaPO₄, pH 6.5, 150 mM NaCl, 0.05% SDS, and 0.01% NaN₃.

In some embodiments, the antigenic determinant and recombinant MHC polypeptide are incubated at about room temperature (for example, at 22-25° C.) to about 75° C. for about 1 to 72 hours. In some examples, the antigenic determinant and recombinant MHC polypeptide are contacted at about 37° C. to about 75° C., for example about 37° C. to 70° C., about 45° C. to about 65° C., or about 50° C. to about 60° C. In some examples, the reaction temperature is about 37° C. to about 60° C. (such as about 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., or 60° C.). In some examples, the reaction time is about 1 hour to 84 hours, such as about 2 hours to 72 hours, about 24 hours to 60 hours, or about 36 hours to 50 hours. In other examples, the antigenic determinant and recombinant MHC polypeptide are contacted for about 1 hour, 2 hours, 3 hours, 6 hours, 12 hours to about 72 hours or more (such as about 12 hours, 16 hours, 18 hours, 24 hours, 36 hours, 48 hours, 50 hours, 55 hours, 60 hours, 72 hours, or more). In one non-limiting example, the antigenic determinant and recombinant MHC polypeptide are contacted at about 37° C. for about 60 hours.

Also disclosed herein are kits for producing the disclosed compositions including a recombinant MHC polypeptide (such as a β1α1 polypeptide or an α1α2 polypeptide) covalently linked to an antigenic determinant by a disulfide linkage. In some embodiments the kit includes a recombinant MHC polypeptide or a nucleic acid encoding a recombinant MHC polypeptide (for example a nucleic acid encoding a recombinant MHC polypeptide in an expression vector) and a solution including one or more components for formation of a disulfide bond (such as a solution described above). In one example, the solution includes about 0.05% SDS and has a pH of about 6.5. In a particular example, the solution includes 100 mM NaPO₄, pH 6.5, 150 mM NaCl, 0.05% SDS, and 0.01% NaN₃.

In additional embodiments, the kit also includes one or more antigenic determinants (such as one or more antigenic peptides). Antigenic peptides can be selected by one of skill in the art and include, but are not limited to those disclosed in Section IID, above. The kit can include additional components, such as cells for expression of a nucleic acid (for example, bacterial or eukaryotic cells), additional buffers (e.g., dialysis buffers), and/or instructions for carrying out methods for producing the composition including a recombinant MHC polypeptide and an antigenic determinant covalently linked by a disulfide bond.

The following examples are provided to illustrate certain particular features and/or embodiments. These examples should not be construed to limit the disclosure to the particular features or embodiments described.

### EXAMPLES

### Example 1

### Materials and Methods

Design, Expression, Production and Purification of rIAg7:

General methods for the design, cloning and expression of recombinant T cell receptor ligands (RTLs), have been previously described (Burrows et al., Protein Eng. 12:771-778, 1999; Chang et al., J. Biol. Chem. 276:24170-24176, 2001; Huan et al., J. Chem. Technol. Biotechnol. 80:2-12, 2005; Fontenot, et al., J. Immunol. 177:3874-3883, 2006; all of which are hereby incorporated by reference). Murine I-A^{s}-derived RTL400 (Offner et al., J. Immunol. 175:4103-4111, 2005) was used as template for constructing the recombinant IAg7 (RTL450-series) genes.

Pairs of oligo-primers specifically designed to modify the template were synthesized and used to generate rIAg7 (RTL450; SEQ ID NO: 4). The gene was directionally ligated into pET21d(+) vector using NcoI and XhoI restriction enzymes (Novagen, Gibbstown, N.J.) and transformed into Nova blue E. coli host (Novagen) for positive colony selection. Primary sequence of the constructs was confirmed by DNA sequencing. Plasmid constructs with confirmed sequences were then transformed into the E. coli strain BL21 (DE3) (Novagen) for expression. Expression, purification and refolding of these proteins followed procedures described previously (Hausmann et al., J. Exp. Med. 189:1723-1734, 1999). In brief, BL21 (DE3) cells containing the plasmid construct of interest were grown in 1-liter cultures to mid-logarithmic phase (OD600=0.6-0.8) in Luria-Bertani (LB) broth containing carbenicillin (50 mg/ml) at 37° C. Recombinant protein production was induced by addition of 0.5 mM isopropyl β-D-thiogalactoside. After incubation for 3 hours, the cells were harvested by centrifugation and stored at −80° C. before processing. All subsequent manipulations of the cells were at 4° C. The cell pellets were resuspended in ice-cold PBS, pH 7.4, and sonicated for 4×20 seconds with the cell suspension cooled in a salt/ice/water bath. The cell suspension was then centrifuged, the supernatant fraction poured off, and the cell pellet resuspended and washed three times in PBS, and then resuspended in 20 mM ethanolamine/6 M urea, pH 10, for 4 hours. After centrifugation, the supernatant containing solubilized rIAg7 was collected and stored at 4° C. until purification.

Purification of rIAg7 involved concentration by fast protein liquid chromatography ion-exchange chromatography using Source 30Q anion exchange media (Pharmacia Biotech/GE Lifesciences, Piscataway, N.J.) in an XK26/20 column (Pharmacia Biotech) charged with buffer B (20 mM ethanolamine pH 10.0, 6 M urea, 2 M NaCl) and then equilibrated with buffer A (buffer B minus NaCl). Approximately 100 ml lysate sample was loaded at 1.5-2.0 ml/min. Protein was eluted using a step gradient (105 ml 1% B, 75 ml 2% B, 120 ml 3% B, 70 ml 4% B), followed by a linear gradient (130 ml 4% to 100% B) and then cleared with 23 ml 100% B, with a flow rate of 5 ml/min. Fractions containing rIAg7 were collected based on analysis of fractions by SDS-PAGE, pooled, and dialyzed extensively against buffer C (6 M urea, 20 mM ethanolamine, pH 10, 200 mM NaCl). For purification to homogeneity a finish step using size-exclusion chromatography on Superdex 75 media (Pharmacia Biotech) in an HR16/50 column (Pharmacia Biotech) in buffer C was used. Fractions containing purified rIAg7 were pooled and diluted to 0.1 mg/ml, and rIAg7 was refolded by extensive dialysis at 0.1 mg/ml against 20 mM Tris, pH 8.5. Protein was then concentrated to 1 mg/ml for short-term storage (4° C.) or snap-frozen in liquid N₂ for long-term storage at −80° C. The final yield of purified rIAg7 (about 90% pure as estimated by SDS-PAGE) varied between 15 and 30 mg/L of bacterial culture.

Redox Capture Conditions:

A mixture of 10:1 peptide:rIAg7 at 200 μg/ml, 100 mM NaPO₄ pH 6.5; 150 mM NaCl; 0.05% SDS; and 0.01% NaN₃ was produced. After inspecting the mixture for any precipitation, reactions were incubated at 37° C. for 60 hours. Analysis of ability of redox capture conditions to facilitate peptide capture by MHC was performed as follows: 20 μl aliquots at various time points were mixed with an equal volume of 2× electrophoresis sample buffer (1% glycerol, 500 mM Tris, 0.2% SDS, and bromophenol blue at pH 8.0). Samples were then placed on ice for 30 minutes, heated at 90° C. for 6 minutes with or without β-mercaptoethanol and separated by 10-20% SDS-PAGE followed by Coomassie Blue staining to visualize the proteins. Proteins were quantified by densitometry scanning using Molecular Imager FX 5 and Quantity One software (Bio-Rad, Hercules, Calif.).

Animals:

C57BL/6 male mice 7-8 weeks of age were obtained from Jackson Laboratories (Bar Harbor, Me.). Gamma interferon-inducible lysosomal thiol reductase (GILT) knockout mice on the C57BL/6 background were also obtained (Maric et al., Science 294:1361-1365, 2001). The mice were housed in the Animal Resource Facility at the Portland Veterans Affairs Medical Center (Portland, Oreg., USA) in accordance with institutional guidelines. The study was conducted in accordance with National Institutes of Health guidelines for the use of experimental animals, and the protocols were approved by the Institutional Animal Care and Use Committee.

Antigens:

Human recombinant MOG (rhMOG) is described in Bettadapura et al (J. Neurochem. 70:1593-1599, 1998). Synthetic human hMOG-35-55 (MEVGWYRSPFSRVVHLYRNGK; SEQ ID NO: 5), mouse MOG (mMOG) (MEVGWYRPPFSRVVHLYRNGK; SEQ ID NO: 6), insulin B:9-23 (SHLVEALYLVCGERG; SEQ ID NO: 3) as well as shortened and cysteine-substituted variants were synthesized by Genscript Inc. (Piscataway, N.J.).

Induction of Active EAE and Treatment with RTL551:

Mice were immunized with 100 μg of rhMOG peptide or 100 μg of mMOG-35-55 peptide in an equal volume of complete Freund's adjuvant containing 2 mg/ml heat-killed Mycobacterium tuberculosis (MTb). All mice were also injected with 75 and 200 ng pertussis toxin intraperitoneally on days 0 and 2 relative to immunization. The mice were assessed for signs of experimental autoimmune encephalomyelitis (EAE) according to the following scale: 0, normal; 1, limp tail or mild hindlimb weakness; 2, moderate hindlimb weakness or mild ataxia; 3, moderately severe hindlimb weakness; 4, severe hindlimb weakness or mild forelimb weakness or moderate ataxia; 5, paraplegia with no more than moderate forelimb weakness; and 6, paraplegia with severe forelimb weakness or severe ataxia or moribund condition. At the onset of clinical signs of EAE (days 10-13 when the clinical scores were ≧2), mice were divided into two groups and treated with 100 μl of 20 mM Tris-HCl as controls or with 100 μl of 1 mg/ml I-A^{b}-derived rIAb (RTL550; “empty”), RTL551 (rIAb with genetically encoded MOG-35-55 amino terminal extension), or RTL550-Cys-MOG (rIA^{b}/MOG) intravenously, along with antihistamine for 8 days. No effect of antihistamine has been observed on EAE induction and progression in SJL/J (Huan et al. J. Immunol. 172:4556-4566, 2004) or C57BL/6 mice. Administration of a molar equivalent dose of free peptide, PLP-139-151 to SJL/J and MOG-35-55 to C57BL/6 mice with antihistamine had no significant clinical benefit to the mice with EAE as compared to untreated mice. Mice were monitored for changes in disease score and were boosted with the treatments as indicated until they were euthanized for ex vivo analyses.

### Example 2

### Disulfide Capture of an Antigenic Peptide

The design and characterization of human DR-, DP- and DQ-, murine I-A^{b}, I-A^{s}, and Lewis rat RT1.B-derived single chain constructs, termed recombinant T cell receptor ligands (RTLs), have been previously described. RTL constructs have the ability to modulate T cell behavior (Burrows et al., J. Immunol. 167:4386-4395, 2001), and have utility in vitro and in vivo in various autoimmune disorders including EAE (an animal model for human multiple sclerosis), chronic beryllium disease, uveitis (Adamus et al., Invest. Ophthalmol. Vis. Sci. 47:2555-2561, 2006) and stroke (Subramanian et al., Stroke 40:2539-2545, 2009).

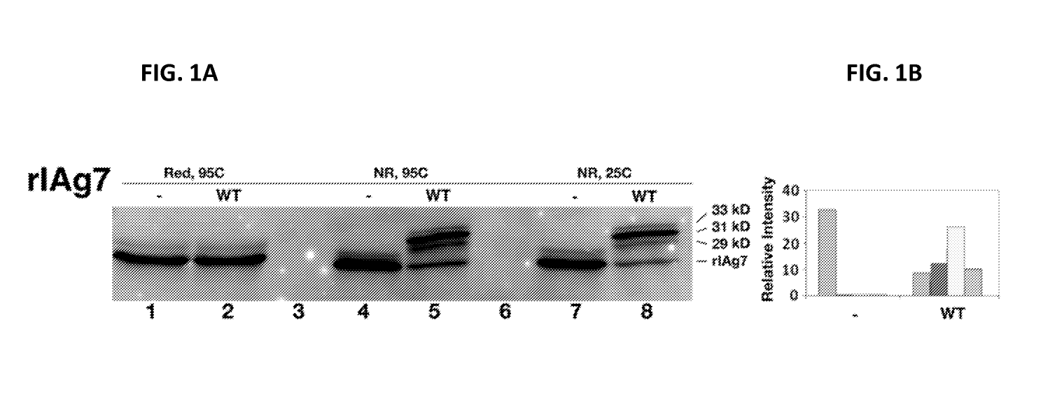

A recombinant form of IAg7 (rIAg7) comprising the β1 and α1 domains of the MHC class II molecule expressed as a single polypeptide (rIAg7; RTL450-series) was designed. IAg7-derived constructs are readily produced in E. coli, are well-behaved in aqueous buffers, and bind antigenic peptides. All MHC class II β1 domains comprise a disulfide bond between cysteine 17 and cysteine 79. Insulin B:9-23 is an antigenic peptide that binds IAg7 and comprises a cysteine residue at position 19. Upon subjecting rIAg7 to disulfide capture conditions in the presence of Insulin B:9-23 (described in Example 1) and then subjecting the sample to electrophoresis under non-reducing conditions, a number of bands of higher apparent molecular weight appeared. The higher molecular weight bands did not appear when the sample was subjected to electrophoresis under reducing conditions (FIG. 1). The higher molecular weight bands were only present when Insulin B:9-23 was added to rIAg7 under disulfide capture conditions and were due to cysteine 19 of the B:9-23 peptide becoming covalently tethered to rIAg7 via disruption of the C17-C79 disulfide bond. IAg7/peptide complexes (WT) migrated as higher molecular weight species (29 and 31 kD species) than the empty rIAg7 molecules.

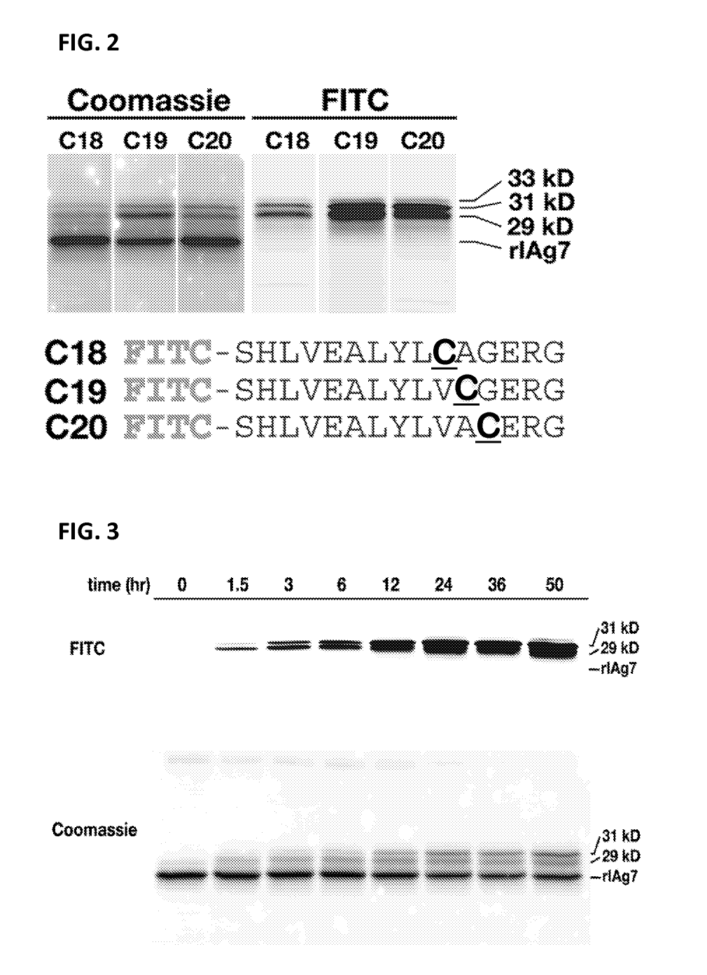

Amino-terminal FITC-coupled B:9-23 derivatives were generated and used to measure peptide binding and rates at which the various higher molecular weight bands appeared were characterized. The most efficiently disulfide captured insulin B:9-23 peptide was the wild-type sequence with Cys at position 19 (FIG. 2). Insulin B:9-23 variants in which the cysteine was moved to position 18 (SHLVEALYLCAGERG; SEQ ID NO: 7) or 20 (SHLVEALYLVACERG; SEQ ID NO: 8), were also disulfide captured, but with significantly less efficiency (FIG. 2).

### Example 3

### Time Course of Disulfide Capture by rIAg7

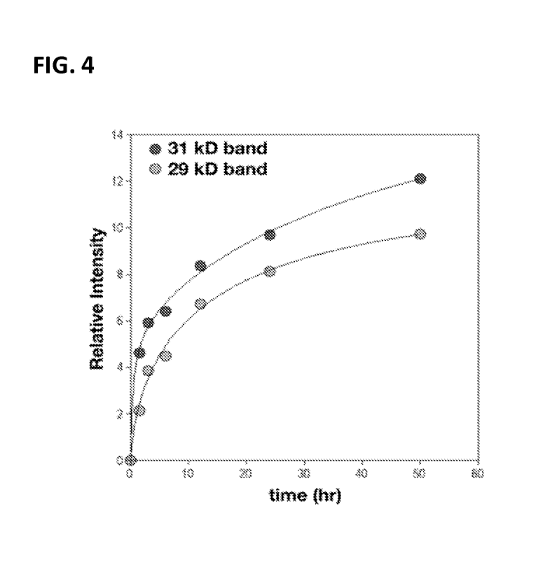

Coomassie stained bands (29 kD and 31 kD, as indicated) were quantified to determine an initial rate of capture (FIG. 3). Insulin B:16-23 peptide (FITC-YLVCGERG; SEQ ID NO: 1) and rIAg7 were mixed (10:1, peptide:rIAg7) in 100 mM NaPO₄, pH 6.5, 150 mM NaCl, 0.05% SDS, and 0.01% NaN₃ and allowed to incubate for the indicated time. Densitometry results are shown in FIG. 4.

A peptide in which the cysteine of B:9-23 was modified to an alanine (SHLVEALYLVAGERG; SEQ ID NO: 9) was not disulfide captured. Additionally, amino-terminal truncated peptide variants of B:9-23 were tested for their ability to be disulfide-captured. The shortest peptide that that could be efficiently captured was the FITC-labeled 8-mer insulin B:16-23 comprising the C-terminal 8 amino acids of B:9-23. This peptide had a half-maximal capture rate of about 1 hour.

### Example 4

### Analysis of Disulfide Capture Using Mass Spectrometry

A combination of proteolytic digestion and mass spectrometry was used to more clearly determine how the insulin B:9-23 peptide was captured, as well as the chemistry of the capture. Primary sequences were used to predict peptides and disulfide cross-linked species of peptides that would be obtained following tryptic digestion of rIAg7. The mass-to-charge ratios (m/z) of the predicted peptides were calculated. Complete tryptic digestion was predicted to yield peptides of interest that would have unique m/z values (Table 1), suggesting that mass spectrometry could reveal the nature of the disulfide capture.

TABLE 1
Mass and charge of potential tryptic digest fragments of interest

| Peptide species | Sequence | m | z | m/z | m/z (+IAA) |
|---|---|---|---|---|---|
| rIAg7 | | | | | |
| 15-25 | GECYFTNGTQR | 1274.5 | 1 | 1275.5 | 1332.5 |
| | (SEQ ID NO: 10) | | 2 | 638.3 | 666.8 |
| 73-80 | AELDTACR | 877.4 | 1 | 878.4 | 935.4 |
| | (SEQ ID NO: 2) | | 2 | 439.7 | 468.2 |
| (15-25)-(73-80) | GECYFTNGTQR | 2149.9 | 2 | 1076.0 | — |
| | \| | | 3 | 717.6 | — |
| | AELDTACR | | 4 | 538.5 | — |
| Insulin B:9-23 | SHLVEALYLVCGERG | 1644.8 | 2 | 823.4 | 851.9 |
| | (SEQ ID NO: 3) | | 3 | 549.3 | 568.3 |
| | | | 4 | 412.2 | 426.5 |
| rIAg7(15-25)- | GECYFTNGTQR | 2917.3 | 2 | 1459.7 | — |
| Ins(9-23) | \| | | 3 | 973.4 | — |
| (C17-C19) | SHLVEALYLVCGERG | | 4 | 730.3 | — |
| rIAg7(73-80)- | AELDTACR | 2520.2 | 2 | 1261.1 | — |
| Ins(9-23) | \| | | 3 | 841.1 | — |
| (C79-C19) | SHLVEALYLVCGERG | | 4 | 631.1 | — |

Table 1 shows predicted tryptic fragments of rIAg7 and insulin B:9-23, including monoisotopic masses (m), possible charge states (z) and m/z ratios of trypsin digest fragments of interest of rIAg7, insulin B:9-23, and potential disulfide cross-linked species. Upon carboxyimidomethylation with iodoacetamide (IAA) of a free cysteine residue, if available, mass would be increased by 57 units. Monoisotopic masses were calculated using a web browser interface calculator (prospector.ucsf.edu/). Note that carboxy-terminal glycine is not cleaved from insulin B:9-23 peptide.

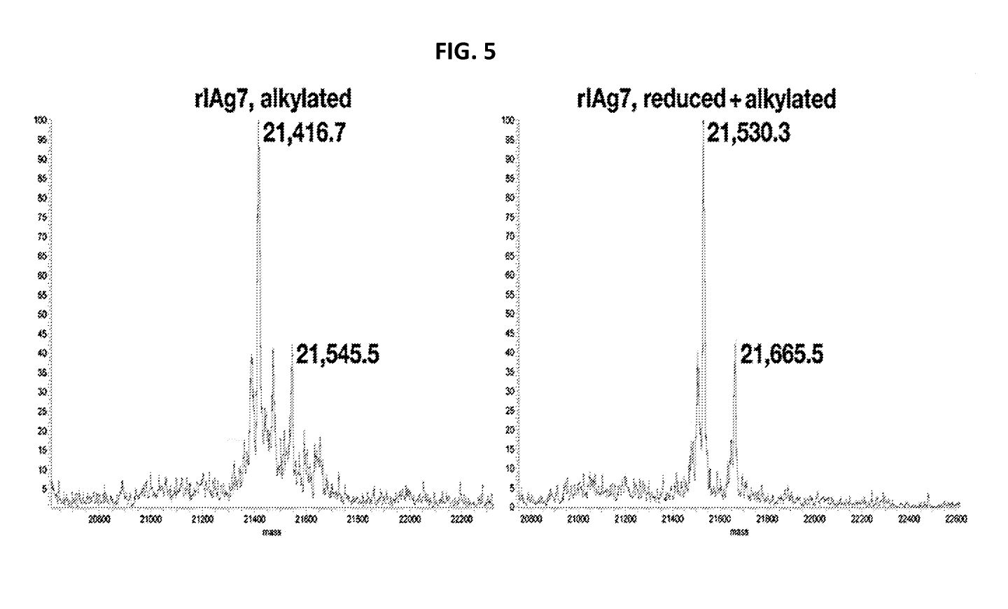

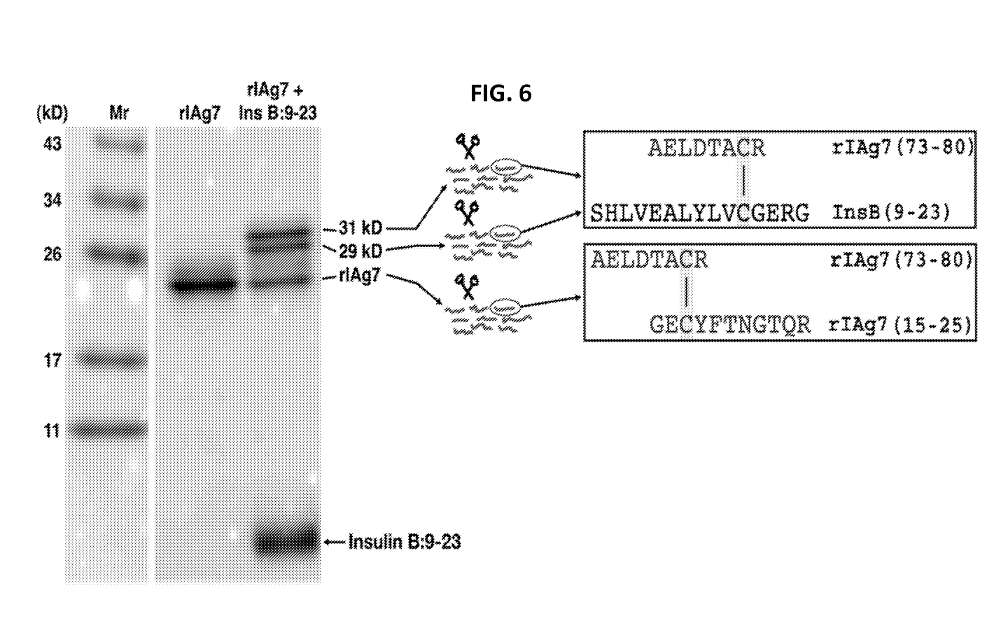

Whole mass measurements confirmed that rIAg7 contained an intact internal disulfide bond (FIG. 5). Insulin B:9-23 was incubated with rIAg7 under redox capture conditions and further disulfide exchange reactions were quenched by addition of IAA. Samples were then boiled and separated under non-reducing conditions by 10-20% SDS-PAGE. After separation by electrophoresis, gel slices corresponding to rIAg7 (about 26 kD), and higher molecular weight bands at 29 kD and 31 kD were excised from the lanes containing rIAg7 loaded with the insulin B:9-23 peptide. The gel slices were digested with trypsin and analyzed by MS. An overview of the process and results is shown in FIG. 6.

Peptide fragments with mass-to-charge (m/z) ratios consistent with the disulfide-linked tryptic fragment containing the intact C17-C79 disulfide bond were readily observed in non-reduced rIAg7 samples (Table 2). Tryptic digestion of reduced and alkylated rIAg7 contained peptide fragments with m/z ratios that corresponded to the 15-25 (GECYFTNGTQR; SEQ ID NO: 10) and 73-80 (AELDTACR; SEQ ID NO: 2) cysteine-alkylated peptide fragments (FIG. 6). The non-reduced 29 kD and 31 kD bands both contained peptide fragments with m/z's corresponding to the 73-80 peptide from rIAg7 disulfide bonded to the insulin B:9-23 peptide (FIG. 6). Peptides with m/z values consistent with linkage at Cys-17 were also observed. The data suggest preferential disulfide capture and insertion of Cys-19 of the insulin B:9-23 peptide into rIAg7 at Cys-79 at least at equilibrium time points (about 50 hour capture).

The amino acid 73-80 tryptic peptide of rIAg7 linked to insulin B:9-23 was readily detected. Tryptic fragments from various samples were analyzed using Xcalibur® software (Thermo-Scientific, Waltham, Mass.) with the goal of determining what mixed disulfide species of interest could be detected following incubation of the insulin B:9-23 peptide with rIAg7. The reduced, unalkylated +2 ion associated with the rIAg7 73-80 was readily observed.

TABLE 2
Actual tryptic peptide fragments from digestion
of rIAg7 disulfide linked to B:9-23.

| | peptide species | | m/z |
|---|---|---|---|
| Sample | detected | | detected |
| rIAg7 | GECYFTNGTQR | (15-25) | 1076.68 |
| non-reduced, | \| | | |
| alkylated | AELDTACR | (73-80) | 718.65 |
| | | | 539.26 |
| rIAg7 | GECYFTNGTQR | (15-25) | 667.57 |
| reduced, | (SEQ ID NO: 10) | | |
| alkylated | AELDTACR | (73-80) | 440.03^{#} |
| | (SEQ ID NO: 2) | | 468.70 |
| | | | 312.80 |
| | | | 234.90 |
| 29 kD band | AELDTACR | (73-80) | |
| rIAg7/Insulin | \| | | |
| B:9-23 mix | SHLVEALYLVCGERG | (InsB:9-23) | 841.68 |
| non-reduced, | | | 631.51 |
| alkylated | | | |
| 31 kD band | AELDTACR | (73-80) | |
| rIAg7/Insulin | \| | | |
| B:9-23 mix | SHLVEALYLVCGERG | (InsB:9-23) | 841.48 |
| non-reduced, | | | 631.60 |
| alkylated | | | |

### Example 5

### Disulfide Capture of Peptides by RTL Derived from Other Class II MHC Molecules

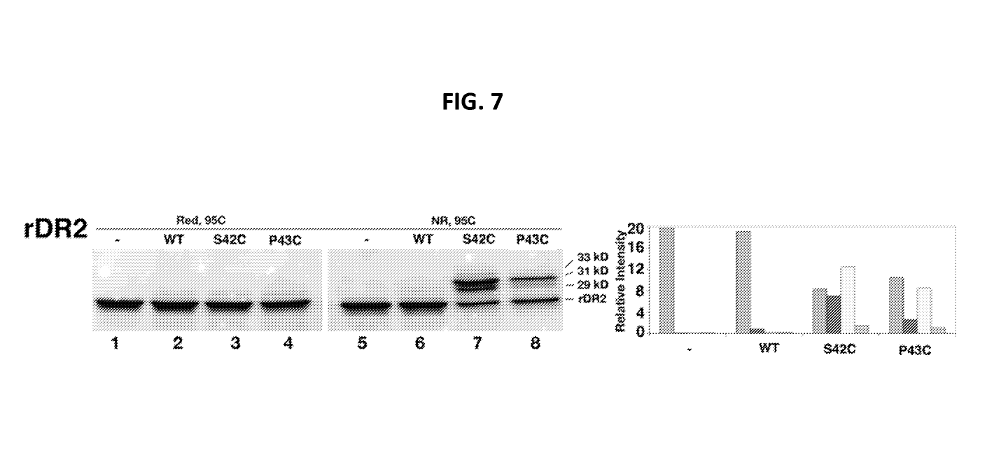

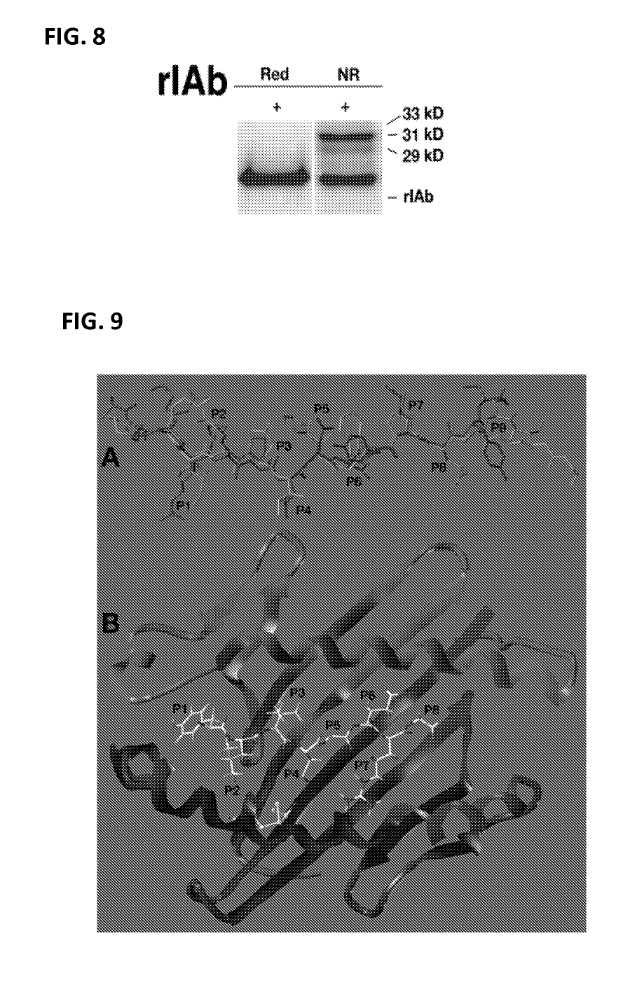

Peptides with single cysteine substitutions at the P4 pocket position and appropriate anchor residues for binding different MHC alleles were generated and have resulted in disulfide capture results of such antigenic peptides by recombinant DR2 (FIG. 7), recombinant I-Ab (FIG. 8), and recombinant DR4 RTLs. The efficiency of disulfide capture depended on the location of the cysteine substitution in the peptide to be captured, how well the peptides fit the binding motif of the MHC allele used and the redox state of the peptides.

Recombinant human DR2 (SEQ ID NO: 11) was loaded with wild type MOG35-55 peptide, which does not include a cysteine residue (SEQ ID NO: 5); the MOG35-55 S42C variant (MEVGWYRCPFSRVVHLYRNGK; SEQ ID NO: 12), which was engineered to comprise a cysteine instead of the serine at position 42; or the MOG35-55 P43C variant (MEVGWYRSCFSRVVHLYRNGK; SEQ ID NO: 13) which was engineered to comprise a cysteine instead of the proline at position 43. rDR2 loaded with unmutated MOG35-55 (WT) did not form any higher apparent molecular weight species and the S42C variant more efficiently formed higher apparent molecular weight species than the P43C variant (FIG. 7). This indicates that the register of peptide binding to rDR2, similar to rIAg7, is a factor that affects the efficiency of capture.

Recombinant murine I-A^{b} (SEQ ID NO: 14) was loaded with S45C mutated mouse MOG35-55 (MEVGWYRPPFCRVVHLYRNGK; SEQ ID NO: 15). Higher apparent molecular weight species formed under reducing conditions (FIG. 8). Some peptides, such as PLP-139-151 (HCLGKWLGHPDKF; SEQ ID NO: 16) formed cysteine-coupled homodimeric peptides instead of interfering with the C17-C79 disulfide bond of the β1 domain of MHC class II. This may be due to the PLP peptide sequence or may have been an artifact of the particular peptide preparation.

### Example 6

### Design of RTL with Disulfide Captured Peptides

Naturally processed peptides from murine H2-IAg7 and human HLA-DQ8 have been used to define a 9-mer core sequence motif with conserved chemical features (Wing et al., Immunol. 106:190-199, 2002; Levisetti et al., Int. Immunol. 15:1473-1483, 2003; Suri et al., J. Clin. Invest. 115:2268-2276, 2005). While the 9-mer core sequence suggested a complex binding motif for IAg7 and DQ8 diabetogenic class II MHC molecules, one clear outcome of sequencing naturally processed peptides supported by crystallographic structural analysis of IAg7 and DQ8 was that both MHC class II alleles favor peptides containing acidic amino acids toward their carboxyl-terminus, with many peptides isolated from both alleles containing runs of double or triple acidic residues toward the C terminus (Wing et al., Immunol. 106:190-199, 2002; Suri et al., J. Clin. Invest. 115:2268-2276, 2005). Structural characterization of IAg7 and DQ8 has clearly documented the unique features of the P9 pocket, and an acidic p9 residue appears to allow formation of an ion pair with Arg76 of the alpha-chain of IAg7, stabilizing the peptide:MHC complex (Corper et al., Science 288:505-511, 2000; Latek et al., Immunity 12:699-710, 2000; Lee et al., Nat. Immunol. 2:501-507, 2001; Yoshida et al., J. Clin. Invest. 120:1578-1590, 2010). A key difference between the IAg7 and DQ8 structures is the P4 pocket, in which DQ8 appears to favor large residues (Lee et al., Nat. Immunol. 2:501-507, 2001). An alignment of insulin B:9-23 peptides in the conventional binding register suggests Tyr16 occupies the P4 pocket of IAg7 as is the case for insulin B:9-23 when bound to HLA-DQ8 structure determined to 3A resolution (PDB accession #1JK8) ((Lee et al., Nat. Immunol. 2:501-507, 2001). However, modeling studies strongly suggest that alternative unconventional binding motifs are also plausible, in particular, one in which Tyr-16 of insulin B:9-23 occupies the P1 pocket of IAg7 and the carboxyl terminus of the peptide contributing acidic contacts in the P9 pocket. This unconventional binding register places C19 in the P4 pocket, at an appropriate distance from the C17-C79 intra-chain disulfide bond that is conserved in all MHC class II beta chains (C15-C79 in full-length MHC class II) so that disulfide capture by insertion into this highly conserved disulfide bond would be possible (FIG. 9).

Many of the details that support this unconventional binding register can be explained by the crystal structures of IAg7 with bound peptides {1ESO, IAg7+GAD-207-220 (YEIAPVFVLLEYVT; SEQ ID NO: 17) (Corper et al., Science 288:505-511, 2000); 1F3J, IAg7+HEL-11-25 (AMKRHGLDNYRGYSL; SEQ ID NO: 18) (Latek et al., Immunity 12:699-710, 2000). Three points are noted. First, the P1 pocket of IAg7 is the largest pocket and is only partially occupied by a hydrophobic isoleucine and three buried water molecules in the 1ESO structure containing the GAD-207-220 peptide (Corper et al., Science 288:505-511, 2000). Both hydrophilic and hydrophobic residues form the surface of the P1 pocket. These include hydrophilic alpha-chain His24 and beta chain Asn82, Thr86, and Glu87, and hydrophobic alpha chain residues Tyr8, Leu31, Phe32, Trp43, Ile52, and Phe54. This diverse set of residues permits a broad specificity in the type and size of the P1 residue, including accommodation of insulin B Tyr-16. Second, the P4 pocket, while small, is hydrophobic in character, and only partially occupied by a valine residue. The additional space could accommodate Cys-19 of insulin B:9-23 as well as slightly larger hydrophobic residues such as leucine or isoleucine. Third, two orientations appear possible for the P9 side chain in IAg7. One points downward into the peptide groove proper and the other points sideways. The shallowness of the P9 pocket suggests that only small side chains (glycine, alanine, and possibly serine) or even short peptides that end at p8 with the carboxyl-terminus providing a carboxyl group, can be accommodated in a downward orientation. The unique sideways orientation observed in the IAg7 structure (Corper et al., Science 288:505-511, 2000) could even accommodate medium to large side chains, although the positively charged environment would favor negatively charged residues. Thus, structural data supports the unconventional register suggested by the data disclosed herein, allowing the possibility that IAg7 could accommodate the insulin B:9-23 peptide with Tyr-16 occupying the P4 pocket.

### Example 7

### Recombinant IAg7 with Disulfide Captured Peptide Treats NOD Mice

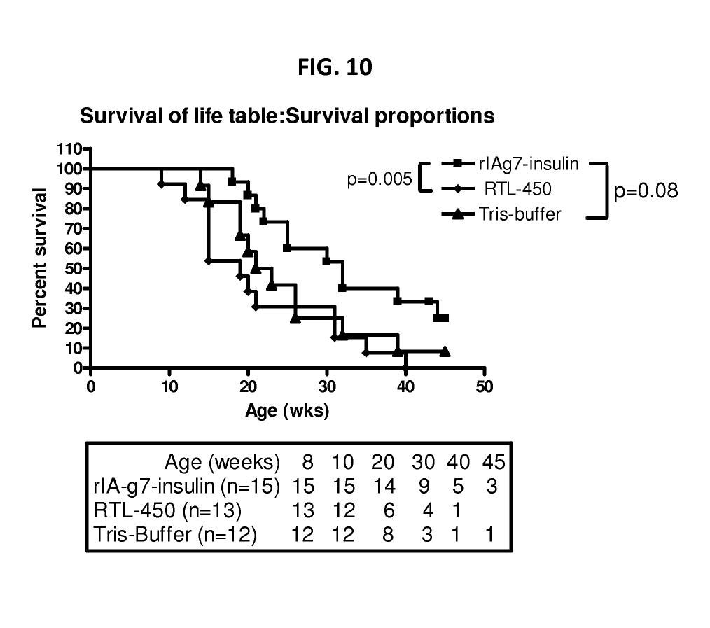

Non-obese diabetic (NOD) mice were treated with recombinant IAg7 with disulfide captured B:9-23 peptide, RTL450 (“empty” rIAg7), or vehicle. Survival was then measured. The mice treated with rIAg7 with disulfide captured B:9-23 showed better survival than both of the other groups (FIG. 10).

### Example 8

### Recombinant DR2 with Disulfide Captured Peptide Treats EAE Mice

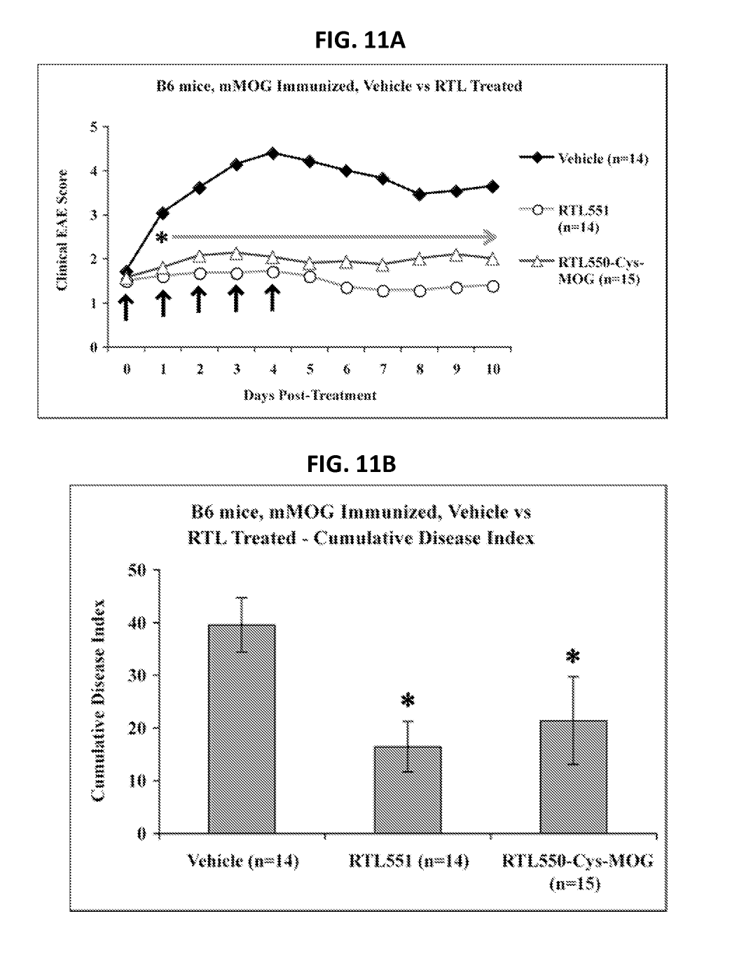

EAE was induced in mice, which were then treated with RTL550 with disulfide captured MOG 35-55 peptide, RTL551 (rIAb with genetically encoded MOG-35-55 amino terminal extension), or vehicle. Clinical EAE score was then measured. The mice treated with RTL550 with disulfide captured MOG 35-55 (RTL-Cys-MOG) had a similar disease course and cumulative disease index compared to mice treated with RTL551 (FIGS. 11A and B; Table 3).

TABLE 3
EAE course in RTL-treated mice

| Group | Incidence | Onset | Peak | Mortality | CDI |
|---|---|---|---|---|---|
| Vehicle | 14/14 | 11.9 ± 0.9 | 4.6 ± 0.4 | 0 | 39.6 ± 5.2 |
| RTL551 | 14/14 | 12.0 ± 1.0 | 2.0 ± 0.7* | 0 | 16.5 ± 4.7* |
| RTL-550- | 15/15 | 12.1 ± 1.1 | 2.7 ± 1.0* | 0 | 21.4 ± 8.3* |
| Cys-MOG | | | | | |

*p < 0.05 vs. vehicle;
CDI, cumulative disease index

### Example 9

### Treatment of GILT Knockout Mice with Disulfide Captured Peptide

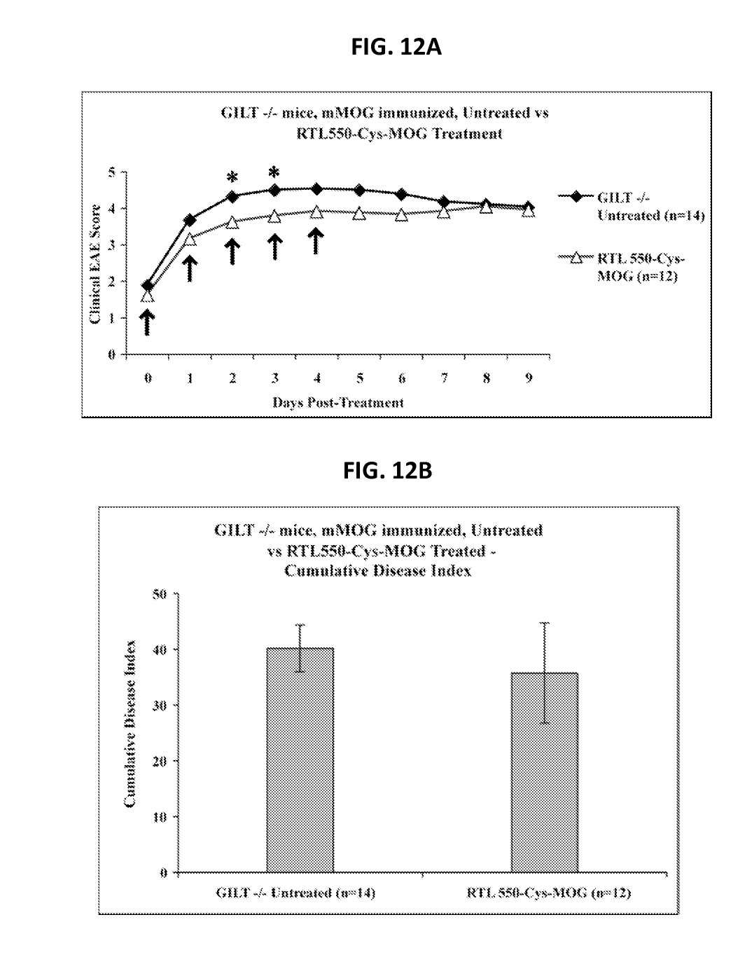

EAE was induced in C57BL/6 GILT−/− mice. Mice were treated with vehicle or RTL550 with disulfide captured MOG35-55 peptide (RTL550-Cys-MOG) and EAE score was measured. Treatment with RTL550 with disulfide captured MOG35-55 did not significantly improve EAE score compared to vehicle-treated mice (FIGS. 12A and 12B). Disease course in treated and untreated mice is shown in Table 4.

TABLE 4
EAE course in GILT −/− mice

| Group | Incidence | Onset | Peak | Mortality | CDI |
|---|---|---|---|---|---|
| Untreated | 14/14 | 10.3 ± 1.3 | 4.7 ± 0.5 | 0 | 40.1 ± 4.2 |
| RTL-550- | 12/12 | 10.5 ± 1.5 | 4.4 ± 1.1 | 0 | 35.8 ± 9.0 |
| Cys-MOG | | | | | |

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A composition comprising:
an isolated recombinant MHC polypeptide comprising covalently linked first and second domains, wherein:
the first domain is a mammalian MHC class II β1 domain and the second domain is a mammalian MHC class II α1 domain and wherein the amino terminus of the second domain is covalently linked to the carboxyl terminus of the first domain and wherein the MHC class II molecule does not include an α2 or a β2 domain; or
the first domain is a mammalian MHC class I α1 domain and the second domain is a mammalian MHC class I α2 domain, and wherein the amino terminus of the second domain is covalently linked to the carboxyl terminus of the first domain and wherein the MHC class I molecule does not include an α3 domain; and
an antigenic determinant covalently linked to the first domain of the recombinant MHC polypeptide by a disulfide bond.

2. The composition of claim 1, wherein the disulfide bond comprises a disulfide bond between a cysteine residue in the antigenic determinant and a cysteine residue in the first domain of the recombinant MHC polypeptide.

3. The composition of claim 2, wherein the first domain of the recombinant MHC polypeptide comprises a β1 domain and the cysteine residue comprises cysteine 17, cysteine 79, or a combination thereof.

4. The composition of claim 1, wherein the covalent linkage between the first domain and the second domain of the recombinant MHC polypeptide comprises a peptide linker.

5. The composition of claim 4, wherein the peptide linker is at least 6 amino acids in length.

6. The composition of claim 1, wherein the antigenic determinant comprises a peptide antigen.

7. The composition of claim 6, wherein the antigenic determinant comprises 8 to 35 amino acids.

8. The composition of claim 1, wherein the antigenic determinant comprises MOG35-55, insulin B:9-23, or PLP139-151.

9. The composition of claim 2, wherein the cysteine residue in the antigenic determinant comprises a non-naturally occurring cysteine, the cysteine residue in the first domain of the recombinant MHC polypeptide comprises a non-naturally occurring cysteine, or a combination thereof.

10. The composition of claim 1, wherein the recombinant MHC polypeptide has reduced potential for aggregation in solution.

11. The composition of claim 10, wherein the recombinant MHC polypeptide comprises a DR2 MHC β1α1 polypeptide comprising substitution of one or more hydrophobic residues with a polar or charged residue, wherein the one or more residues are selected from V102, I104, A106, F108, and L110 of SEQ ID NO: 11, thereby having reduced aggregation in solution as compared to an unmodified recombinant MHC polypeptide.

12. The composition of claim 1, further comprising a pharmaceutically acceptable carrier.

13. A method of treating or inhibiting a disorder selected from the group consisting of an autoimmune disease, a retinal disease, uveitis, stroke, and cognitive impairment or neuropsychiatric disorder induced by substance addiction in a subject, comprising administering an effective amount of the composition of claim 1 to the subject, thereby treating or inhibiting the disorder.

14. The method of claim 13, wherein the autoimmune disease is selected from the group consisting of multiple sclerosis, type I diabetes, rheumatoid arthritis, celiac disease, psoriasis, systemic lupus erythematosus, pernicious anemia, myasthenia gravis, optic neuritis, and Addison's disease.

15. A method of treating or inhibiting type I diabetes in a subject comprising administering to the subject an effective amount of a composition comprising an isolated recombinant MHC polypeptide comprising covalently linked first and second domains, wherein the first domain is a mammalian MHC class II β1 domain and the second domain is a mammalian MHC class II α1 domain and wherein the amino terminus of the second domain is covalently linked to the carboxyl terminus of the first domain and wherein the MHC class II molecule does not include an α2 or a β2 domain and an antigenic determinant covalently linked to the first domain of the recombinant MHC polypeptide by a disulfide bond, thereby treating or inhibiting type I diabetes in the subject.

16. The method of claim 15, wherein the antigenic determinant comprises insulin.

17. The method of claim 15, wherein the antigenic determinant comprises insulin B:9-23 or insulin B:16-23.

18. The method of claim 17, wherein the insulin B:9-23 comprises the amino acid sequence of any one of SEQ ID NOs: 1, 3, and 7-9.

19. A method of producing a composition, comprising:
contacting an antigenic determinant with an isolated recombinant MHC polypeptide comprising covalently linked first and second domains, wherein:
the first domain is a mammalian MHC class II β1 domain and the second domain is a mammalian MHC class II α1 domain and wherein the amino terminus of the second domain is covalently linked to the carboxyl terminus of the first domain and wherein the MHC class II molecule does not include an α2 or a β2 domain; or
the first domain is a mammalian MHC class I α1 domain and the second domain is a mammalian MHC class I α2 domain, and wherein the amino terminus of the second domain is covalently linked to the carboxyl terminus of the first domain and wherein the MHC class I molecule does not include an α3 domain; and
under conditions sufficient for a disulfide bond to form between the antigenic determinant and the recombinant MHC polypeptide, thereby producing the composition.

20. The method of claim 19, wherein the conditions sufficient for a disulfide bond to form comprise contacting the antigenic determinant and the recombinant MHC polypeptide at 37° C. for 60 hours in 100 mM NaPO₄, pH 6.5, 150 mM NaCl, 0.05% SDS, and 0.01% NaN₃.

21. A kit for producing the composition of claim 1, comprising:
a recombinant MHC polypeptide or a nucleic acid encoding the recombinant MHC polypeptide; and
a solution comprising one or more components for providing conditions sufficient for formation of a disulfide bond between the recombinant MHC polypeptide and an antigenic determinant.

22. The kit of claim 21, wherein the solution comprises 100 mM NaPO₄, pH 6.5, 150 mM NaCl, 0.05% SDS, and 0.01% NaN₃.

23. The kit of claim 21, further comprising an antigenic determinant.

24. A composition produced by the method of claim 19.
